(19) United States
(12) Patent Application Publication
(10) Pub. No.: US 2018/0140239 A1
(43) Pub. Date: May 24, 2018

(54) HETEROGENEOUS ANALYTE SENSOR APPARATUS AND METHODS

(71) Applicant: GlySens Incorporated, San Diego, CA (US)

(72) Inventors: Joseph Lucisano, San Diego, CA (US); William Markle, Laguna Niguel, CA (US); Robert Gazdzinski, San Diego, CA (US)

(21) Appl. No.: 15/359,406

(22) Filed: Nov. 22, 2016

(51) Int. Cl. A61B 5/1486; A61B 5/145; A61B 5/1495; A61B 5/00

(52) U.S. Cl. CPC A61B 5/14865; A61B 5/14532; A61B 5/1495; A61F 2002/009; A61B 5/6861; A61B 5/0031; A61B 2560/0219; A61B 5/7264

(57) ABSTRACT

Implantable sensor and associated receiver apparatus, and methods of manufacturing, implantation, and use. In one embodiment, the sensor apparatus is a heterogeneous glucose sensor, including hydrogen peroxide-based detector elements and oxygen-based detector elements. The sensor apparatus utilizes one (type) of the detector elements to confirm the accuracy of, and/or calibrate, the second (type of) detector element, so as to among other things enable the second type of detector to operate more robustly, and/or for a longer period without external calibration and/or explants. In one variant, the heterogeneous detector types are contained within a common biocompatible implantable housing. In another variant, the first type of detector (e.g., oxygen based) is disposed within a separable module that can operate independently of (or mechanically and/or electrically interface with) the second type of detector. In yet another variant, an external receiver compatible with both the first and second types is disclosed.

### RELATED APPLICATIONS

This application is related to co-owned and co-pending U.S. patent application Ser. No. 13/559,475 filed Jul. 26, 2012 entitled “Tissue Implantable Sensor With Hermetically Sealed Housing,” Ser. No. 14/982,346 filed Dec. 29, 2015 and entitled “Implantable Sensor Apparatus and Methods”, Ser. No. 15/170,571 filed Jun. 1, 2016 and entitled “Biocompatible Implantable Sensor Apparatus And Methods”, and Ser. No. 15/197,104 filed Jun. 29, 2016 and entitled “Bio-adaptable Implantable Sensor Apparatus And Methods”, each of the foregoing incorporated herein by reference in its entirety. This application is also related to U.S. patent application Ser. No. 10/719,541 filed Nov. 20, 2003, now issued as U.S. Pat. No. 7,336,984 and entitled “Membrane and Electrode Structure for Implantable Sensor,” also incorporated herein by reference in its entirety.

### GRANT INFORMATION

This invention was made in part with government support under NIH Grant No. DK-77254. The United States government has certain rights in this invention.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### 1. Technical Field

The disclosure relates generally to the field of sensors, therapy devices, implants and other devices (such as those which can be used consistent with human beings or other living entities for in vivo detection and measurement or delivery of various solutes), and in one exemplary aspect to methods and apparatus enabling the use of such sensors and/or electronic devices for, e.g. monitoring of one or more physiological parameters, including through use of a novel membrane structure and/or other components and characteristics.

### 2. Description of Related Technology

Implantable electronics is a rapidly expanding discipline within the medical arts. Owing in part to significant advances in electronics and wireless technology integration, miniaturization, performance, and material biocompatibility, sensors or other types of electronics which once were beyond the realm of reasonable use in vivo in a living subject can now be surgically implanted within such subjects with minimal effect on the recipient subject, and in fact many inherent benefits.

One particular area of note relates to blood glucose monitoring for subjects, including those with so-called “type 1” or “type 2” diabetes. As is well known, regulation of blood glucose is impaired in people with diabetes by: (1) the inability of the pancreas to adequately produce the glucose-regulating hormone insulin; (2) the insensitivity of various tissues that use insulin to take up glucose; or (3) a combination of both of these phenomena. Safe and effective correction of this dysregulation requires blood glucose monitoring.

Currently, glucose monitoring in the diabetic population is based largely on collecting blood by “fingersticking” and determining its glucose concentration by conventional assay. This procedure has several disadvantages, including: (1) the discomfort associated with the procedure, which should be performed repeatedly each day; (2) the near impossibility of sufficiently frequent sampling (some blood glucose excursions require sampling every 20 minutes, or more frequently, to accurately treat); and (3) the requirement that the user initiate blood collection, which precludes warning strategies that rely on automatic early detection. Using the extant fingersticking procedure, the frequent sampling regimen that would be most medically beneficial cannot be realistically expected of even the most committed patients, and automatic sampling, which would be especially useful during periods of sleep, is not available.

Implantable glucose sensors have long been considered as an alternative to intermittent monitoring of blood glucose levels by the fingerstick method of sample collection. These devices may be fully implanted, where all components of the system reside within the body and there are no through-the-skin (i.e. percutaneous) elements, or they may be partially implanted, where certain components reside within the body but are physically connected to additional components external to the body via one or more percutaneous elements. The operability of one such fully implanted sensor has been demonstrated as a central venous implant in dogs (Armour et al., Diabetes, 39:1519 1526 (1990), incorporated herein by reference in its entirety). Although this sensor provided direct recording of blood glucose, which is most advantageous for clinical applications, the described implantation at a central venous site poses several risks and drawbacks, including risk of blood clot formation and vascular wall damage. An alternative that does not present such risks to the user is to implant the sensor in a “solid” tissue site and to relate the resulting signal to blood glucose concentration.

Typical sensors implanted in solid tissue sites measure the concentration of solutes, such as glucose, in the blood perfusing the microcirculation in the vicinity of the sensor. Glucose diffuses from nearby capillaries to the sensor surface. Because such diffusion occurs effectively only over very small distances, the sensor responds to the substrate supply only from nearby blood vessels. Conversely, solutes that are generated in the locality of the sensor may be transported away from the sensor's immediate vicinity by the local microvasculature. In either case, access to and/or association with the local microcirculation may influence the sensor's response.

Optical glucose sensors are known in the prior art. Schultz and Mansouri disclosed one such version of an optical sensor (J. S. Schultz and S. Mansouri, “Optical Fiber Affinity Sensors,” Methods in Enzymology, K. Mosbach, Ed., Academic Press, New York, 1988, vol. 137, pp. 349-366). A variety of other optical techniques including optical coherence tomography, near infrared spectroscopy, Raman spectroscopy, and polarimetry have been tried and failed. Light-based systems using either absorption of light, or emission of light when glucose is “excited” by light have not proven to be accurate since there is no specific light absorption or emission spectrum for glucose. Furthermore, numerous other chemicals or interfering substances in the blood overlap in spectrum with glucose, causing optical methods to be insufficiently specific for glucose monitoring.

A number of electrochemical glucose sensors have also been developed, most of which are based on the reaction catalyzed by the enzyme glucose oxidase. One such configuration involves the use of glucose oxidase to catalyze the reaction between glucose and oxygen to yield gluconate and hydrogen peroxide. The hydrogen peroxide is either detected directly, or can be further decomposed by a second enzyme, e.g. catalase, in which case the sensor measures oxygen consumption. In order for glucose oxidase based sensors to function properly, the presence, in the vicinity of the enzyme, of excess molecular oxygen relative to molecular glucose is necessary. However, this requirement gives rise to a sensor design problem related to “oxygen deficit,” since the concentration of oxygen in bodily tissues is significantly less than that of glucose.

For example, the typical concentration of glucose in the blood is about 4 to about 20 mM, whereas a typical concentration of oxygen in blood plasma may be only about 0.05 to about 0.1 mM. Oxygen concentrations in other tissue fluids may be even lower. As the chemical reaction, and thus, the sensor signal, is limited by the reactant that is present in the sensor's reaction zone at the lowest concentration, an implanted sensor of simple construction would remain limited by oxygen, and would therefore be insensitive to the metabolite of interest (e.g. glucose). Thus, there is a need for differential control of the permeability of the sensor diffusion device (e.g., “membrane”) to restrict or modulate the flux of the metabolite of interest (e.g. glucose), and provide a stoichiometric equivalent or excess of oxygen in the reaction zone. The sensor incorporating such a membrane can then be sensitive to the metabolite of interest over the physiologic range. Also, for successful functioning of the implanted sensor, the membrane material exposed to the bodily tissue must further be biocompatible, or elicit a favorable response from the body. Several membrane solutions have been proposed to date.

One such solution has been through the use of macroporous or microporous membranes to ratio the diffusion of oxygen and glucose to the sensing elements, such as that set forth in U.S. Pat. No. 4,759,828 to Young, which discloses use of a laminated membrane with an outer microporous membrane having a pore size of 10 to 125 A (Angstrom) to limit the diffusion of glucose molecules. However, one problem with the use of a macroporous or microporous membrane relates to exposure of the sensing element of the sensor to the environment of the body, which can result in “fouling” or other deleterious effects. Another solution is disclosed in U.S. Pat. No. 4,671,288 to Gough, which describes a cylindrical device, implantable in an artery or vein, which is permeable to glucose only at an end of the device, and with both the curved surface and end permeable to oxygen. In vascular applications, the advantage is direct access to blood glucose, leading to a relatively rapid response. However, a major disadvantage of vascular implantation is the possibility of eliciting blood clots or vascular wall damage, as noted supra.

U.S. Pat. No. 5,660,163 to Schulman discloses another solution through use of a silicone rubber membrane containing at least one “pocket” filled with glucose oxidase in a gelatinous glucose- and oxygen-permeable material located over a first working electrode, such that the length of the “pocket” is a multiple of its thickness to optimize the linearity between current and the glucose concentration measurement. However, because the long axis of the “pocket” is oriented parallel to the electrode surface, this design may be less amenable to miniaturization for tissue implantation, and may suffer from yet other disabilities relating thereto.

Still further, another solution has been to utilize a composite membrane that is hydrophilic and also contains small hydrophobic domains to increase the membrane's overall gas solubility, giving rise to differential permeability of glucose and oxygen (e.g. U.S. Pat. Nos. 4,484,987 and 4,890,620 to Gough). However, one salient disadvantage of this approach relates to the requirement that the amount of hydrophobic polymer phase must be relatively large to allow for adequate oxygen permeability. This substantially reduces the hydrophilic volume available for enzyme inclusion sufficient to counter inactivation during long-term operation.

Another alternative is described in U.S. Pat. No. 4,650,547 to Gough, which discloses a “stratified” structure in which the electrode was first overlaid with an enzyme-containing layer, and second with a non-glucose-permeable membrane. The resulting structure is permeable to oxygen over a large portion of the surface of the membrane, whereas glucose can only reach the enzyme through the “edge” of the device, thus regulating access of the reactants to the enzyme.

A significant concern in the context of e.g., implantable solid tissue devices is the so-called “tissue response”, wherein the host's physiology proximate to the implanted sensor is irritated or adversely stimulated into an antibody-modulated or other response which can be deleterious to the operation of the implanted device, especially over longer periods of time. The process of implantation (i.e., creation of a wound) and the presence of a device (i.e., a foreign body) within living tissue cause early host reactions (e.g., within two to four weeks of implantation) that generally include: (i) blood-biomaterial interaction, (ii) provisional matrix formation, (iii) acute inflammation, (iv) chronic inflammation, (v) foreign body reaction (FBR), and (vi) fibrosis/fibrous capsule development (Anderson, James. “Biological Responses to Materials.” Annu. Rev. Mater. Res. 31(2001): 81-110.). Each of these phases of wound healing has a cascade effect, including release of specific bio-chemicals (e.g., mitogens, chemoattractants, cytokines, growth factors, etc.) and migration of specific wound healing-associated cells (e.g., neutrophils, macrophages, fibroblasts, foreign body giant cells, etc.) to the implant site, which leads to subsequent phases, and eventually adaptation to or rejection of the implanted device.

In some cases, although the living tissue adapts to the implanted device, the wound healing process may render the device non-functional (or at very least reduce its functionality and/or accuracy), thereby negating any benefit to the patient. For example, in implanted devices that depend on diffusive transport of solutes to or from the bloodstream (e.g. implanted chemical sensors), such responses can negatively impact device operation due to an increase in mass transfer resistance between the bloodstream and active portions of the device surface resulting from an FBR-mediated development of fibrous tissue surrounding the device. The FBR also can complicate explants of the implanted device (due to, e.g., the FBR causing significant encapsulation of the implanted device, thereby increasing its effective size when explanted), and result in yet other disabilities. Thus, accounting for (and minimizing) the FBR remains an important consideration for nearly all implanted devices. Some prior art solutions for implantable sensors have attempted to use layers external to the sensing enzyme region to actively modulate or eliminate the FBR. Such approaches have typically used materials for such layer(s) which are designed to encourage blood vessel growth and perfusion in the vicinity of the sensor or into the layer(s), which is undesirable, because such modulated responses are often not predictable and furthermore may not be sustainable for extended durations.

An illustration of the final phases of a typical wound healing 100 response are depicted in the example of FIG. 1, showing an implanted object 102 and surrounding host tissue 104. In the FBR phase 106 of wound healing, tissue repair cells 108 (e.g., macrophages, foreign body giant cells, etc.) are recruited to the surface of the object 102 and the surrounding tissue 104. Subsequently, in the fibrosis phase 110, the object 102 undergoes fibrous encapsulation by granulation tissue and/or connective tissue 112.

Biocompatibility of a medical device, such as e.g., an implantable sensor, may be defined as the ability of the device to perform as intended with an appropriate host wound healing response, while minimizing the magnitude and duration of the wound healing response. Factors that affect biocompatibility may include, inter alia, extent of injury (e.g., amount of tissue removal, size of incision, etc.) resulting from the implantation process, integrity of basement membrane structures during and after implantation, material compositions of the device, surface properties of the device, dimensions of the device, exposure of tissues to electrical and/or chemical components (including byproducts) of the device, motion and/or migration of the device in the implant site, and ability to function under at least a minimal degree of granulation tissue formation, FBR, and fibrosis.

Traditionally, “solid tissue” sensors (including the aforementioned glucose sensors) are implanted within the living subject at a generally superficial layer or level of the tissue, so as to (i) mitigate tissue trauma resulting from the surgical implantation procedure, and (ii) mitigate interference from interposed solid tissue to the propagation of electromagnetic radiation (e.g., wireless transmissions to and from the implant). Specifically, historically larger implants require a larger volume within the solid tissue of the recipient, and hence placing the larger implant further down into the layers of tissue, etc. residing below the epidermis requires a larger incision, possibly including through various blood vessels, basement membranes, and/or other features and possibly requiring removal of some solid tissue to accommodate the volume of the implant, thereby likely extending duration and intensity the host's wound healing response.

In one specific example, some conventional glucose sensors monitor glucose via detection of hydrogen peroxide, which is a product of glucose reaction with oxygen catalyzed by the glucose oxidase (GOX) enzyme. Hydrogen peroxide is widely regarded as a cytotoxic agent that can lead to cell death and tissue necrosis in excess concentrations, and also can stimulate the wound healing response. In another example, some glucose sensors (peroxide-based or otherwise) may be configured such that enzyme-embedded membranes are directly exposed to the host blood and tissue, which may trigger an immunogenic response. In even another example, some implants may be comprised of materials that increase duration and/or intensity of wound healing. Likewise, electrical circuitry and/or electrochemical processes associated with an implanted or partly implanted device may trigger a similar immunogenic response in the host. For instance, electrical currents and potentials associated with an electrochemical sensor can, if sufficiently proximate to the host's tissue, induce varying degrees of the aforementioned tissue response, which is likewise undesired.

Additionally, motion and/or migration of an implanted device may exacerbate the chronic inflammation phase of wound healing. Prolonged chronic inflammation is also associated with increased FBR and fibrosis, and may lead to implant rejection and require extraction or “explant” (i.e., removal of the sensor). The explant process generally becomes more difficult and traumatic to the tissue if there is significant FBR and fibrosis, which may involve the growth of significant amounts of connective tissue around the implanted sensor over time.

It is recognized that at least minimal levels of FBR and fibrosis (i.e., end stages of tissue response) are normal to the wound healing process. The FBR is characterized by the formation of foreign body giant cells, which adhere to surfaces of the device and stimulate fibrosis (i.e., encapsulation by fibrous connective tissue with a decreased density of capillary blood vessels relative to undisturbed tissue) of the device in an attempt to isolate the implant from the local tissue environment. The materials, form, and topography of the surface of the implanted device, as well as the degree and duration of previous stages of wound healing may all affect the FBR and fibrosis processes. When the FBR is minimized, there is generally increased regeneration of normal tissue, and replacement of tissue by the fibrous capsule is decreased. In some conventional implanted sensors, even normal degrees of FBR and fibrosis may obstruct the sensing components, thereby rendering the device non-functional and necessitating replacement (i.e., explant of the current device and implant of a new device), which may reinitiate the wound healing process.

Moreover, blood vessel vascularization and “ingrowth” into portions of an implanted device (such as an implanted sensor) may occur in certain prior art applications, effectively bonding the device (at least in certain areas) to the host, and thereby precluding a clean separation of the device from the surrounding FBR-induced encapsulation during device explant. Some prior art solutions for implantable sensors have attempted to use layers external to the sensing region to actively modulate or eliminate the FBR; see, e.g., U.S. Pat. No. 6,558,321 to Burd, et al. entitled “Systems and methods for remote monitoring and modulation of medical devices,” which describes use of a porous material on the exterior of the sensor's enzyme membrane element. Such approaches have typically used materials for such layer(s) which are designed to encourage blood vessel growth and perfusion in the vicinity of the sensor or into the layer(s), which is undesirable, because such modulated responses are often not predictable and furthermore may not be sustainable for extended durations.

Accuracy is also an important consideration for implanted analyte sensors, especially in the context of blood glucose monitoring. Hence, ensuring accurate measurement for extended periods of time (and minimizing the need for any other confirmatory or similar analyses) is of great significance. Further, having adequate dynamic range for the implanted sensor is important, particularly as it relates to accuracy. Simply stated, the implanted sensor device should be able to accurately measure the target analyte over the entire normal (or even abnormal) range of values that may be encountered within the host's physiology.

In known conventional sensors, response and accuracy can be adversely affected by a FBR or other tissue response in the region of the analyte sensor as noted above; this effect can be exacerbated the longer the sensor is left implanted. Specifically, as the FBR or tissue response proceeds over time, the mechanical relationship between an implanted sensor device and the host's tissue in the immediate area of implantation (including micro-perfusion within blood vessels adjacent to the sensor) can significantly change due to movement between the tissue (and the microvascular structures therein which provide communication between the device and the body's circulatory system) and the device surface, thereby potentially degrading the accuracy and/or reliability of the sensor device. Notwithstanding, the host tissue needs to be maintained in close physical contact with the detector or sensor of the implanted device, in order for the sensor to operate properly (e.g., by enabling the blood glucose molecules to migrate into the sensor for utilization therein). Hence, there is somewhat of a “catch-22” involved; any effective sensor will need to be implanted at a site with sufficient available blood glucose (delivered via blood vessels or microvasculature of the host in that area) and maintain close physical contact with the tissue at that site for proper and accurate sensor operation, yet such close contact (including even the act of implantation) can trigger a tissue response which can be deleterious to the accuracy and operation of the sensor. Sensors relying on the diffusion of glucose are particularly susceptible to variations in tissue response and encapsulation, since these factors directly affect the rate and magnitude of glucose diffusion from the capillaries to the implanted sensor element.

Lastly, many conventional implantable devices are sufficient only for relatively short-term implantation due to expiration or exhaustion of one or more components of the device (as well as the aforementioned degradation of accuracy/response due to effects of the FBR). In this case, similar to devices obstructed by FBR and fibrosis, the devices may necessitate frequent replacement (i.e., explant of the current device and implant of a new device), which may reinitiate the wound healing process.

The Assignee hereof has more recently developed improved methods and apparatus for implanting and measuring blood glucose level which overcome the aforementioned disabilities with the prior art; see, inter alia, U.S. patent application Ser. Nos. 13/559,475, 14/982,346, 15/170,571, and 15/197,104 previously incorporated herein. However, Assignee has further recognized that (i) there are a significant number of such prior art devices currently in use and commercially available, including a large body of supporting research and product research and development; and (ii) if certain of the disabilities associated with such prior art technology are effectively addressed (including especially the comparatively rapid calibration “drift” and/or need for frequent confirmatory fingersticks or other similar approaches), such prior art devices may have utility in certain applications.

### SUMMARY

The present disclosure satisfies the foregoing needs by providing, inter alia, improved apparatus for accurately sensing analyte levels within a living subject, including for extended periods of time without explant, and methods of manufacturing and operating the same.

In one aspect of the disclosure, an implantable blood analyte sensing device is disclosed. In one embodiment, the device includes: at least one first analyte detector of a first type; at least one second analyte detector of a second type differing from the first type; and logic in signal communication with the at least one first analyte detector and the at least one second analyte detector.

In one variant, the logic is configured to utilize signals generated by the at least one second detector to enable at least one of: (i) confirmation of a blood analyte level estimate obtained from signals generated by the at least one first analyte detector; and/or (ii) calibration of a blood analyte level estimate obtained from signals generated by the at least one first analyte detector.

In one implementation, the at least one first analyte detector of the first type comprises a peroxide-based blood glucose detector, and the at least one second analyte detector of the second type comprises a non-peroxide based blood glucose detector, with the at least one second analyte detector having a glucose oxidase (GOX) and catalase enzyme matrix.

In another implementation, the non-peroxide based blood glucose detector includes: a biocompatible housing having a size and shape suitable for implantation in a body; a plurality of non-peroxide based analyte detector elements; circuitry operatively connected to the plurality of detector elements and configured to process at least a portion of signals generated by one or more of the detector elements to produce processed signals; and an electrical power source operatively coupled to at least the circuitry and configured to provide electrical power thereto. The non-peroxide based detector elements each further comprise a membrane configured for direct contact with a biological tissue of a host being after implantation of the sensor device, the membrane at least partly permeable to diffusion of the blood glucose therethrough, yet which is configured to frustrate blood vessel ingrowth.

In another aspect, a method of operating an implantable sensing device is disclosed. In one embodiment, the method includes: utilizing at least a first detector element of the sensing device to determine blood analyte level within a host being; and utilizing at least a second detector element of the sensing device to confirm the determined blood analyte level.

In one variant, the utilizing at least a first detector element of the sensing device comprises using at least one peroxide-based detector element, and the utilizing at least a second detector element comprises using at least one oxygen-based detector element, and the confirmation includes determining that the determined blood analyte level is within a prescribed range of values, the prescribed range based at least in part on a blood analyte level determined by the at least second detector element.

In one implementation, the at least second detector element comprises a plurality of individual detector elements, and the blood analyte level determined by the at least second detector element is determined using at least an algorithm operative to at least weight signals produced by certain of the plurality of individual detector elements over signals produced by others of the plurality.

In another implementation, the weighting of the signals produced by certain of the plurality of individual detector elements over signals produced by others of the plurality comprises weighting based at least on a detected calibration drift of one or more of the plurality over time (which may include both homogeneous and heterogeneous detector weighting approaches).

In a further variant of the method, the utilizing at least a first detector element of the sensing device comprises using at least one oxygen-based detector element, and the utilizing at least a second detector element comprises using at least one peroxide-based detector element on an intermittent or periodic basis only so as to mitigate formation of a foreign body response within the host being.

In another embodiment, the method includes: utilizing at least a first detection enzyme apparatus of the sensing device to determine blood analyte level within a host being; and at least periodically utilizing at least a second detection enzyme apparatus of the sensing device to calibrate the determined blood analyte level. In one variant, the second detection enzyme apparatus comprises an enzyme matrix which is configured to mitigate foreign body response within the host being through elimination of a substance or compound formed within the second detector enzyme apparatus, which is also used within the first detector enzyme apparatus for said determination of blood analyte level.

In a further aspect of the disclosure, a blood analyte sensing device configured for implantation in a living host being is disclosed. In one embodiment, the device includes: at least one first analyte detector of a first type; at least one second analyte detector of a second type differing from the first type; a communications interface; and logic in signal communication with the at least one first analyte detector and the at least one second analyte detector and the communications interface. In one variant, the logic is configured to process signals generated by the at least one first analyte detector and the at least one second analyte detector so as to enable transmission via the communications interface to a receiving apparatus external to the host being.

In one implementation, the at least one first analyte detector of the first type comprises a peroxide-based blood glucose detector, and the at least one second analyte detector of the second type comprises a non-peroxide based blood glucose detector. The processing of the signals generated by the at least one first analyte detector and the at least one second analyte detector enables, inter alia, transmission via an extant communications protocol associated with the at least one first analyte detector.

In another aspect of the disclosure, a blood analyte sensing module configured for implantation in a living host along with another sensing device is disclosed. In one embodiment, the module includes: at least one analyte detector utilizing an enzyme matrix for detection of blood analyte levels; a wireless communications interface; and logic in signal communication with the at least one analyte detector and the wireless communications interface, the logic configured to process signals generated by the at least one analyte detector for transmission via the wireless communications interface to a receiving apparatus external to the host being, the receiving apparatus associated with the another sensing device.

In one implementation, the transmission via the wireless communications interface to a receiving apparatus external to the host being comprises transmission according to a wireless transmission protocol utilized by the first (e.g., peroxide-based) sensing device to transmit signals to the receiving apparatus.

In another variant, the module is configured to physically mate to or integrate with at least a portion of the another sensing device to permit implantation of the module and the another sensing device as a unitary structure.

In another embodiment, the module includes at least one analyte detector utilizing an enzyme matrix for detection of blood analyte levels; a first communications interface; a wireless communications interface; and logic in signal communication with the at least one analyte detector, the first communications interface, and the wireless communications interface, In one variant, the logic is configured to process at least signals generated by the at least one analyte detector for transmission via the wireless communications interface to a receiving apparatus external to the host being, the receiving apparatus associated with the sensing module.

In one implementation, the processing of at least signals generated by the at least one analyte detector for transmission via the wireless communications interface comprises processing at least both: (i) signals generated by the at least one analyte detector; and (ii) signals generated by the another sensing device and received by the module via the first communications interface.

In a further aspect of the disclosure, wireless receiver apparatus configured to receive data from each of: (i) an implanted blood analyte sensing device; and (ii) another blood analyte sensing device, is disclosed. In one embodiment, the receiver apparatus includes a wireless communications interface; and computerized processing logic in signal communication with the wireless communications interface. In one variant, the logic is configured to process at least (i) data generated by the implanted blood analyte sensing device and received via the wireless communications interface; and (ii) data generated by the another blood analyte sensing device and received via the wireless communications interface.

In one implementation, the data received via the wireless communications interface from the implanted blood analyte sensing device are formatted according to a first communications protocol, and the data received via the wireless communications interface from the another blood analyte sensing device are formatted according to a second communications protocol different than the first communications protocol. In another implementation, the data received via the wireless communications interface from the implanted blood analyte sensing device are used to perform at least one of: (i) a calibration of the data received via the wireless communications interface from the another blood analyte sensing device; and/or (ii) confirmation of an accuracy of the data received via the wireless communications interface from the another blood analyte sensing device.

In a further aspect, a fully implantable analyte sensor is disclosed. In one embodiment, the sensor includes: a processing apparatus, one or more hydrogen peroxide-based glucose sensor elements in signal communication with the processing apparatus, and one or more oxygen-based glucose sensor elements in signal communication with the processing apparatus. The one or more hydrogen peroxide-based glucose sensor elements are configured to measure and transmit one or more localized hydrogen peroxide concentration signals and the one or more oxygen-based glucose sensor elements are configured to measure and transmit one or more localized oxygen concentration signals, each of the one or more localized hydrogen peroxide-based signals and the one or more localized oxygen concentration signals being usable by the processing apparatus to estimate a blood glucose concentration.

In one variant, the one or more localized hydrogen peroxide concentration signals are configured as a reference signal for the one or more localized oxygen concentration signals. In another variant, the one or more localized oxygen concentration signals are configured as a reference signal for the one or more localized hydrogen peroxide concentration signals.

In yet another variant, the sensor further includes: a biocompatible housing having a size and shape suitable for implantation in a body, active surfaces of the one or more hydrogen peroxide-based glucose sensor elements and the one or more oxygen-based glucose sensor elements being disposed on an outer surface of the biocompatible housing; circuitry operatively connected to the one or more hydrogen peroxide-based glucose sensor elements and the one or more oxygen-based glucose sensor elements, and configured to process at least a portion of the one or more localized hydrogen peroxide concentration signals and the one or more localized oxygen concentration signals to produce processed signals; data transmission apparatus configured to transmit at least a portion of the processed signals to a receiver (whether inside the body, outside of the body, or combinations thereof) when the implanted sensor is disposed in a tissue environment within the body; and an electrical power source operatively coupled to at least the circuitry and data transmission apparatus and configured to provide electrical power thereto.

In one implementation, the circuitry is configured such that at least a portion of the one or more hydrogen peroxide-based glucose sensor elements and the one or more oxygen-based glucose sensor elements are able to adapt for variations in a biophysical interface between the sensor elements and biological tissue of the body over time, the variations caused at least in part by physiological processes within the body. In one variant, the peroxide-based sensor elements are used as the “primary” elements, being periodically calibrated or corrected by the “secondary” oxygen-based sensors. When degradation or EOL of the primary peroxide-based sensors is detected, they are ignored or deactivated in favor of the oxygen-based sensors, the latter having an appreciably longer in vivo lifetime and functionality.

In still another variant, the sensor further comprises apparatus configured to promote interlock of at least a portion of the plurality of detectors with biological tissue of the body proximate thereto without substantive blood vessel ingrowth. In one implementation, the apparatus configured to promote interlock comprises at least one membrane configured for direct contact with the biological tissue after implantation of the sensor, the at least one membrane at least partly permeable to diffusion of the blood glucose therethrough, yet which is configured to frustrate the blood vessel ingrowth.

In another variant, a non-enzymatic membrane comprising a crosslinked albumin material that is substantially permeable to at least glucose and oxygen present at the outer surface thereof, is used as an interface between at least the non-peroxide enzyme matrix and the host tissue. The non-enzymatic membrane is also placed in contact with a top surface of the enzyme material so as to permit diffusion of the oxygen and glucose into the enzyme material to permit chemical interaction therein, while affording the solid tissue of the host an unreactive “buffer zone,” thereby isolating the host tissue from direct contact with the enzyme material.

In another variant, the response time (at a given oxygen level) of each of the one or more hydrogen peroxide-based glucose sensor elements and the one or more oxygen-based glucose sensor elements is selectable or controllable through control of one or more physical attributes of the non-enzyme membrane and/or its surrounding “spout” structure (e.g., base height, spout height, and/or non-enzyme membrane thickness).

In another variant, each of the one or more hydrogen peroxide-based glucose sensor elements and the one or more oxygen-based glucose sensor elements is capable of measuring blood glucose level both (i) within a prescribed range of response and (ii) within a prescribed time period.

In another variant, each of the one or more hydrogen peroxide-based glucose sensor elements and the one or more oxygen-based glucose sensor elements includes: a substantially enclosed cavity, the substantially enclosed cavity comprising at least one enzymatic substance, and at least one aperture in communication with the cavity; an electrolyte layer; at least one electrode disposed at least partly within or contacting the electrolyte layer; and a non-enzymatic membrane at least partly occluding the aperture, the non-enzymatic membrane comprising a material at least partly permeable to an analyte yet which does not exacerbate an FBR (including fibrous tissue growth). In one implementation, the non-enzymatic membrane comprises a crosslinked albumin-based material, and the each of the one or more hydrogen peroxide-based glucose sensor elements and the one or more oxygen-based glucose sensor elements is configured to utilize chemical interaction between at least an analyte (glucose) and the enzymatic substance to enable generation of an electrical signal at the electrode via the electrolyte layer, the electrical signal relating to a concentration of the analyte in a region external to the cavity and the membrane.

In another embodiment, the sensor is configured to estimate a blood analyte concentration and includes: a processing apparatus, one or more first analyte sensor elements in signal communication with the processing apparatus, and second analyte sensor elements in signal communication with the processing apparatus. The one or more first sensor elements are configured to measure and transmit one or more localized first analyte byproduct or constituent concentration signals and the one or more second analyte sensor elements are configured to measure and transmit one or more second analyte byproduct or constituent concentration signals, each of the one or more localized first analyte byproduct or constituent concentration signals and one or more second analyte byproduct or constituent concentration signals being usable by the processing apparatus to estimate the blood analyte concentration.

In another embodiment, the analyte sensor is configured with heterogeneous sensing range sensor elements. In one embodiment, the sensor includes two or more first sensor elements and two or more second sensor elements capable of measuring blood glucose level in at least one of: (i) different ranges of response, and/or (ii) different times or rates of response. The two or more first sensor elements are configured to detect a first molecule concentration and the two or more second sensor elements are configured to detect a second different molecule concentration. In one variant, each sensor element includes a non-enzymatic membrane element in contact with the solid tissue, and the range of response (at a specified glucose level and a specified oxygen level) of each sensor element is selectable or controllable through control of one or more physical attributes of the membrane and/or its surrounding “spout” structure (e.g., base diameter and and/or spout diameter).

In another aspect, a miniaturized biocompatible implantable sensor is disclosed. In one embodiment, the sensor comprises one or more hydrogen peroxide-based glucose sensing elements and one or more oxygen-based glucose sensing elements disposed on a sensing region thereof, and is fabricated from biocompatible materials and uses biocompatible processes for sensing which advantageously mitigate or eliminate physiological responses from the host (e.g., chronic inflammation, FBR, blood vessel in-growth, and/or fibrosis), while also enabling close physical contact with the host's tissue so as to permit long-term, accurate blood glucose monitoring and easy subsequent explant of the sensor.

In one variant, the sensor is further configured to dynamically accommodate any tissue changes which do occur, algorithmically (e.g., within the control logic of the device). In one particular implementation, the miniaturized size, optimized materials and construction, and adaptive operation of the sensor apparatus enable, inter alia, deeper and less traumatic implantation within the host's solid tissue (and subsequent extraction) and continued operation within the host for extended periods of time.

In another aspect of the disclosure, a method of mitigating a foreign body response (FBR) within a living being while monitoring blood glucose level using an implanted sensor apparatus is described. In one embodiment, the method includes: allowing at least oxygen molecules and glucose molecules from the living being's blood to permeate through a non-enzymatic layer or membrane to each of a first enzyme-containing material and a second enzyme-containing material with which the oxygen molecules and glucose molecules can chemically interact, the chemical interaction enabling the monitoring; and at least mitigating egress of enzymes within the enzyme-containing material outward through the layer or membrane. In one variant, the non-enzymatic material comprises a protein-based (e.g., albumin) substance which is chemically crosslinked, and which does not encourage blood vessel growth into its thickness. In another variant, the first enzyme-containing material and the second enzyme containing material are diffusionally isolated from one another. In one implementation, the first enzyme-containing material is a glucose oxidase enzyme matrix and the second enzyme-containing material is a glucose oxidase and catalase enzyme matrix.

In yet another aspect, a method of configuring an implantable sensing device (including at least one hydrogen-peroxide based glucose sensor element and at least one oxygen-based glucose sensor element) so as to limit tissue response from a living host in which the device is ultimately implanted is disclosed. In one embodiment, the method includes configuring the sensing device to facilitate contact of at least one outer membrane thereof with tissue of the living host when the device is implanted, the facilitating contact comprising (i) enabling tissue response by the living host to substantially cover or encase at least a portion of the at least one outer membrane; and (ii) not encouraging or avoiding vascularization by the living host into the at least one outer membrane.

In one variant, the implantable sensor comprises a glucose sensor, and the enabling tissue response comprises configuring the sensing device such that it is in direct physical contact with the tissue of the living host when implanted so as to facilitate migration of at least blood glucose molecules to the at least one outer membrane, and the not encouraging vascularization comprises configuring the at least one outer membrane to have a pore size on at least an outer surface thereof sufficient to inhibit the vascularization.

In still another aspect, a method of maintaining a position and orientation of an implantable sensor within a living host while also maintaining its operability is disclosed. In one embodiment, the sensor includes a first sensor element and a second different sensor element each configured for sensing an analyte (e.g., glucose), and the method includes: implanting the sensor within a location of the host; enabling a tissue response to the implanted sensor such that tissue of the host proximate the implanted sensor substantially interlocks with the sensing feature; and frustrating vascularization of the tissue into the sensing feature. The substantial interlock with the sensing feature provides mechanical stability to the sensor so as to maintain the position and orientation, minimizing movement between the sensor surface and the tissue adjacent to the sensor without causing any significant “bonding” of the tissue to the sensing feature or sensor body. Minimizing the potential for relative movement or slippage between the sensor surface and the adjacent tissue helps ensure stability of the sensor response characteristics and also avoids exacerbating the FBR from mechanically-induced fibrotic response effects.

In a further aspect, a method of extending the in vivo operating lifetime of an implantable glucose sensor including one or more hydrogen peroxide-based sensing elements and one or more oxygen-based sensing elements is disclosed. In one embodiment, the method includes controlling a level of tissue response and blood vessel vascularization from a host being over time such that close contact between the solid tissue of the host and a sensing region of the implantable device is achieved, yet simultaneously mitigating vascularization into the sensing region and encapsulation of at least the remainder of the sensing apparatus. In one variant, the foregoing control is accomplished via coordination of a plurality of configuration factors, including: (i) electrical insulation of the solid tissue in at least the sensing region of the device, (ii) enzyme insulation of the solid tissue in at least the sensing region of the device; (iii) use of an outer anti-vascularization sensor barrier for at least some of the sensors in the sensing region, and (iv) use of substantially smooth, biocompatible materials for portions of the device outside of the sensing region.

In yet another aspect, methods and apparatus for providing measurement or estimation of blood analyte level (e.g., glucose level) using a heterogeneous sensor apparatus are disclosed. In one embodiment, the sensor apparatus comprises an implantable device with a first sensor type (e.g., peroxide-based) and a second sensor type (e.g., oxygen-based), and the method includes using the first sensor type for a period of time, before using the second sensor type thereafter. In one implementation, the period of time is selected based on one or more operational parameters, such as stability of one or more of the first and second sensor types; e.g., the first sensor type is used until sufficient stability of the output signal of the second sensor type is achieved, at which point the second sensor type is utilized as the primary signal source. In another implementation, no period of time per se is selected, but rather the sensor apparatus is configured to automatically (or under command of an external receiver/control apparatus) switch over to use of the oxygen-based detector elements as the primary signal source when certain parametric criteria are met. Other features and advantages of the present disclosure will immediately be recognized by persons of ordinary skill in the art with reference to the attached drawings and detailed description of exemplary embodiments as given below.

### BRIEF DESCRIPTION OF THE DRAWINGS

FIG. 1 is an illustration of foreign body response and fibrosis phases of a typical wound healing response that may occur after implantation of an object or device.

FIG. 2 is a front perspective view of one exemplary embodiment of a fully implantable biocompatible sensor apparatus according to the present disclosure.

FIGS. 2A-2C are top, bottom, and side elevation views, respectively, of the exemplary sensor apparatus of FIG. 2.

FIG. 3 is a side cross-sectional view of one exemplary detector element of a detector array in a fully implantable sensor apparatus according to the present disclosure.

FIG. 3A is a side cross-sectional view of one exemplary spout region (outer non-enzyme membrane removed) of a detector element of a detector array in a fully implantable sensor apparatus according to one embodiment of the present disclosure.

FIG. 3B is a side cross-sectional view of exemplary adjacent spout regions of example first and second type detector elements of a detector array in a fully implantable sensor apparatus according to one embodiment of the present disclosure.

FIG. 4 is a top elevation view of another exemplary embodiment of the sensor apparatus of present disclosure, wherein multiple sensor/reference pairs with at least partly differing glucose sensing ranges are used on a common device.

FIG. 5 is a functional block diagram of one embodiment of an implantable heterogeneous sensing device and associated receiver apparatus, according to the disclosure.

FIG. 6 is a functional block diagram of another embodiment of an implantable heterogeneous sensing device and associated receiver apparatus according to the disclosure, wherein the device comprises a first portion, and a second “add-on” module that can be mechanically, logically, and/or electrically coupled to the first portion.

FIG. 7 is a functional block diagram of yet another embodiment of an implantable heterogeneous sensing device and associated receiver apparatus according to the disclosure, wherein the receiver is configured to receive signals from each of the heterogeneous detector elements directly.

FIG. 8A is a functional block diagram of one embodiment of a wireless receiver apparatus useful with various aspects of the present disclosure.

FIG. 8B is a functional block diagram of another embodiment of a wireless receiver apparatus useful with various aspects of the present disclosure.

FIG. 8C is a functional block diagram of yet another embodiment of a wireless receiver apparatus useful with various aspects of the present disclosure.

FIG. 9 is a logical flow diagram illustrating one embodiment of a generalized method for use of heterogeneous sensors for measurement of an analyte within a living being.

FIG. 9A is a logical flow diagram illustrating one exemplary implementation of the generalized method of FIG. 9.

FIG. 9B is a logical flow diagram illustrating another exemplary implementation of the generalized method of FIG. 9.

FIG. 9C is a logical flow diagram illustrating yet another exemplary implementation of the generalized method of FIG. 9.

FIG. 10 is a logical flow diagram illustrating one exemplary implementation of the evaluation and correction processes of the method of FIG. 9.

FIG. 11 is a logical flow diagram illustrating one exemplary embodiment of a method for operating a heterogeneous implantable analyte sensor so as optimize its implanted (usable) lifetime.

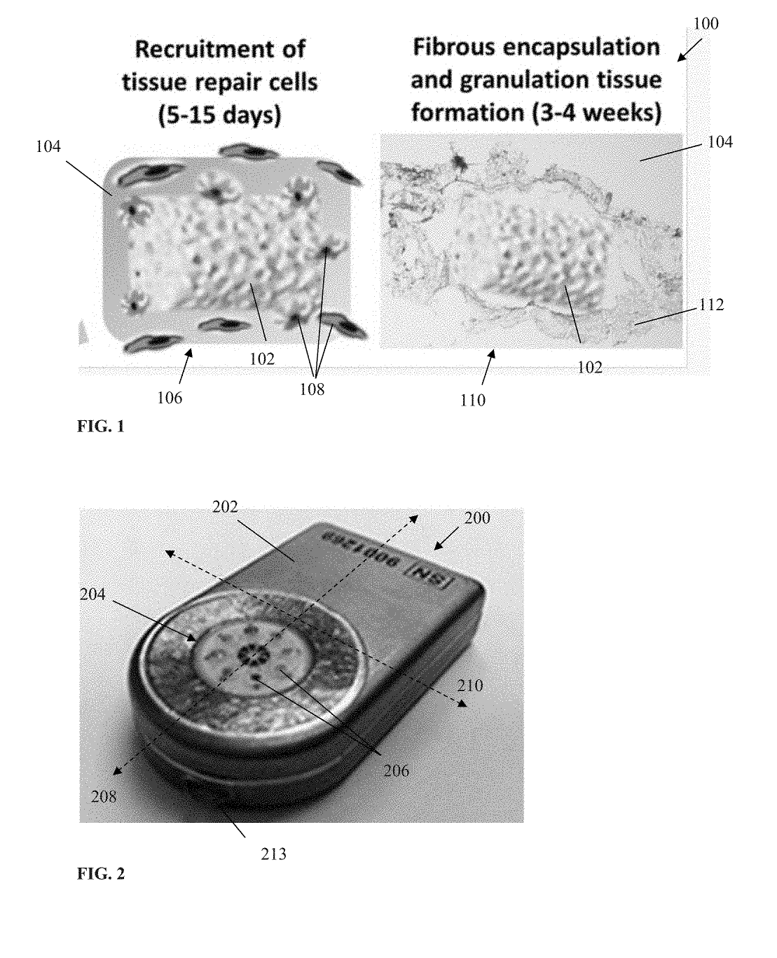

All Figures © Copyright 2015-2016 GlySens Incorporated. All rights reserved.

### DETAILED DESCRIPTION

Reference is now made to the drawings, wherein like numerals refer to like parts throughout.

### Overview

One aspect of the present disclosure leverages Assignee's recognition that there may be circumstances where certain aspects of the prior art approaches may be used consistent with the foregoing improved methods and apparatus, in combination, such that at least some of the above-described disabilities of a given prior art approach can be mitigated or even completely eliminated.

Accordingly, the present disclosure makes use of the combination of certain advantageous aspects of the construction and/or operation of its above-referenced improved solutions with prior art technology (including for example peroxide-based implantable devices and their associated receivers) to provide improvements over the prior art device alone.

In one such implementation, the aforementioned implantable peroxide-based blood glucose sensor is used in conjunction with Assignee's non-peroxide-based detector element(s), the latter acting in a confirmatory or calibration capacity to in effect “second check” and adjust (as necessary) the peroxide sensor, thereby ostensibly extending the interval between other confirmatory processes utilized with the device (e.g., fingersticking), and/or the implant-to-explant interval, hence improving user experience with the device and quality of life.

In one exemplary configuration, the first and second analyte detector element types are used in parallel, and one detector type acts as a reference for the other detector type (e.g., the hydrogen peroxide-based glucose detector element is a reference for the oxygen-based glucose detector element, the oxygen-based glucose detector element is a reference for the hydrogen peroxide-based glucose detector element, etc.). In another example, the first and second analyte detector element types are used in parallel and measurements or readings from both detector element types are used to generate or derive a composite measurement (e.g., via a weighted average). In yet another example, the first and second analyte detector types can be alternately and/or selectively employed depending on specific implantation, use, and/or physiological conditions.

Advantageously, the apparatus and methods of the disclosure enable, inter alia, substantially continuous, long-term and accurate monitoring of blood glucose levels in living beings using the aforementioned implantable sensor apparatus, without the need for prior art “finger sticks,” transcutaneous apparatus worn on external surfaces of the body, or intravenous and other implantable devices, each having their own disabilities as previously described.

The exemplary implementation of the foregoing biocompatible sensor apparatus is also advantageously suitable for “long-term” implantation (e.g., 12-18 months) by virtue of its design and operation, thereby decreasing reoccurrence of injury and repeated inducement of the wound healing response necessitated by expiration and replacement of the device, which can be performed on an outpatient basis by a clinician using only local anesthetic and recovery time from the procedure is minimal.

Moreover, the “imprint” or impression created by the sensor apparatus within the tissue of the host when implanted can be advantageously re-used (whether by a subsequent replacement sensor of the same or similar configuration, or the same sensor apparatus that has been e.g., refitted with a new battery), so that the foreign body response or other deleterious host responses are yet further avoided, and trauma to the host is minimized.

The aforementioned implementation may include one or more features that dynamically adapt operation of the sensor apparatus to the host's tissue response over time, leveraging the observation that any non-mitigated response can be accounted for by the sensor apparatus, such as via signal processing either within, or off-board from, the sensor apparatus while implanted.

The disclosed configuration (including use of the enzyme-free layer) advantageously does not encourage blood vessel ingrowth, which inter alia, enables accurate sensor apparatus operation during periods of extended implantation. As discussed above, by not encouraging such ingrowth, otherwise unstable/unpredictable modulation of FBR to the extent required to encourage blood vessel ingrowth is limited. In one variant, such ingrowth is frustrated (or not encouraged) through selective regulation of pore sizes within the non-enzymatic layer.

Control of response range and/or rate also permits easy “customization” of sensor elements, whether on a per-element, per-element type, or per-sensor apparatus basis. For example, the techniques of the present disclosure allow for ready construction of an implantable sensor apparatus having multiple heterogeneous detector elements with multiple respective ranges of sensitivity and/or rates of detection, thereby extending the dynamic range of the sensor apparatus (both in terms of analyte concentration, co-reactant concentration, and/or time, as desired).

Moreover, in one variant, the various heterogeneous detector elements (e.g., detector elements of the first glucose detector type and the second glucose detector type, and/or detector elements of various configurations for either sensor type according to specified ranges of sensitivity and/or rates of detection) can be selectively switched on/off (even while the sensor apparatus is in vivo), so as to, e.g., accommodate “on the fly” changes to blood glucose concentration or other physiological changes occurring within the host, or to maintain efficacy of the detector elements within a known or desirable range of accuracy or sensitivity. One type of detector can also be prioritized over another, or swapped out, such as e.g., where the performance of one detector type has eroded over time (due to e.g., FBR associated with that particular detector), or loss of some other desirable attribute or performance aspect.

Methods of implantation and methods of manufacturing the aforementioned membranes and sensor elements are also disclosed herein.

### Detailed Description of Exemplary Embodiments

Exemplary embodiments of the present disclosure are now described in detail. While these embodiments are primarily discussed in the context of a fully implantable glucose sensor, such as those exemplary embodiments described herein, and/or those set forth in U.S. Patent Application Publication No. 2013/0197332 filed Jul. 26, 2012 entitled “Tissue Implantable Sensor With Hermetically Sealed Housing;” U.S. Pat. No. 7,894,870 to Lucisano et al. issued Feb. 22, 2011 and entitled “Hermetic Implantable Sensor;” U.S. Patent Application Publication No. 2011/0137142 to Lucisano et al. published Jun. 9, 2011 and entitled “Hermetic Implantable Sensor;” U.S. Pat. No. 8,763,245 to Lucisano et al. issued Jul. 1, 2014 and entitled “Hermetic Feedthrough Assembly for Ceramic Body;” U.S. Patent Application Publication No. 2014/0309510 to Lucisano et al. published Oct. 16, 2014 and entitled “Hermetic Feedthrough Assembly for Ceramic Body;” U.S. Pat. No. 7,248,912 to Gough et al. issued Jul. 24, 2007 and entitled “Tissue Implantable Sensors for Measurement of Blood Solutes;” and U.S. Pat. No. 7,871,456 to Gough et al. issued Jan. 18, 2011 and entitled “Membranes with Controlled Permeability to Polar and Apolar Molecules in Solution and Methods of Making Same;” and U.S. Patent Application Publication No. 2013/0197332 to Lucisano et al. published Aug. 1, 2013 and entitled “Tissue Implantable Sensor with Hermetically Sealed Housing;” PCT Patent Application Publication No. 2013/016573 to Lucisano et al. published Jan. 31, 2013 and entitled “Tissue Implantable Sensor with Hermetically Sealed Housing,” each of the foregoing incorporated herein by reference in its entirety, as well as those of U.S. patent application Ser. Nos. 13/559,475, 14/982,346, 15/170,571, and 15/197,104 previously incorporated herein, it will be recognized by those of ordinary skill that the present disclosure is not so limited. In fact, the various aspects of the disclosure are useful with, inter alia, other types of implantable sensors and/or electronic devices.

Further, while the following embodiments describe specific implementations of e.g., biocompatible peroxide-based and oxygen-based multi-sensor element devices for measurement of glucose, having specific configurations, protocols, locations, and orientations for implantation (e.g., proximate the waistline on a human abdomen with the sensor array disposed proximate to fascial tissue; see e.g., U.S. patent application Ser. No. 14/982,346 filed Dec. 29, 2015 and entitled “Implantable Sensor Apparatus and Methods” previously incorporated herein), those of ordinary skill in the related arts will readily appreciate that such descriptions are purely illustrative, and in fact the methods and apparatus described herein can be used consistent with, and without limitation: (i) in living beings other than humans; (ii) other types or configurations of sensors (e.g., other types, enzymes, and/or theories of operation of glucose sensors, sensors other than glucose sensors, such as e.g., sensors for other analytes such as urea, lactate); (iii) other implantation locations and/or techniques; and/or (iv) devices intended to deliver substances to the body (e.g. implanted drug pumps, drug-eluting solid materials, and encapsulated cell-based implants, etc.); and/or other devices (e.g., non-sensors and non-substance delivery devices).

As used herein, the term “analyte” refers without limitation to a substance or chemical species that is of interest in an analytical procedure. In general, the analyte itself cannot be measured, but a measurement of the analyte (e.g., glucose) can be derived through measurement of chemical constituents, components, or reaction byproducts associated with the analyte (e.g., hydrogen peroxide, oxygen, free electrons, etc.).

As used herein, the terms “biocompatible” and “biocompatibility” refer without limitation to the ability of a medical device or implantable material to perform as intended in the presence of an appropriate host wound healing response and/or other immunogenic responses, while minimizing magnitude and duration of the wound healing (e.g., acute inflammation, chronic inflammation, foreign body reaction (FBR), and fibrosis/fibrous capsule development) and causing no significant harm to the patient.

As used herein, the terms “detector” and “sensor” refer without limitation to a device having one or more elements (e.g., detector element, sensor element, sensing elements, etc.) that generate, or can be made to generate, a signal indicative of a measured parameter, such as the concentration of an analyte (e.g., glucose) or its associated chemical constituents and/or byproducts (e.g., hydrogen peroxide, oxygen, free electrons, etc.). Such a device may be based on electrochemical, electrical, optical, mechanical, thermal, or other principles as generally known in the art. Such a device may consist of one or more components, including for example, one, two, three, or four electrodes, and may further incorporate immobilized enzymes or other biological or physical components, such as membranes, to provide or enhance sensitivity or specificity for the analyte. As used herein, the terms “enzyme free” and “non-enzymatic” include, without limitation, materials that are completely enzyme-free, and materials that are substantially enzyme free (e.g., may have a small percentage of residual or unintentional enzymes).

As used herein the term “membrane” refers without limitation to a substance, layer or element configured to have at least one desired property relative to the aforementioned analyte, such as e.g., a permeability to a given type of analyte, co-reactants, or other substance.

As used herein, the terms “orient,” “orientation,” and “position” refer, without limitation, to any spatial disposition of a device and/or any of its components relative to another object or being, and in no way connote an absolute frame of reference.

As used herein, the terms “top,” “bottom,” “side,” “up,” “down,” and the like merely connote, without limitation, a relative position or geometry of one component to another, and in no way connote an absolute frame of reference or any required orientation. For example, a “top” portion of a component may actually reside below a “bottom” portion when the component is mounted to another device (e.g., host sensor).

As used herein, the terms “wound healing” and “tissue response” refer without limitation to biological processes that occur within a host or patient during and after implantation. The biological processes generally including the following phases: (i) blood-biomaterial interaction, (ii) provisional matrix formation, (iii) acute inflammation, (iv) chronic inflammation, (v) foreign body reaction (FBR), and (vi) fibrosis/fibrous capsule development. Although each phase is generally subsequent the preceding phase, phases maybe overlapping and/or reoccurring.

### Exemplary Implantable Sensor

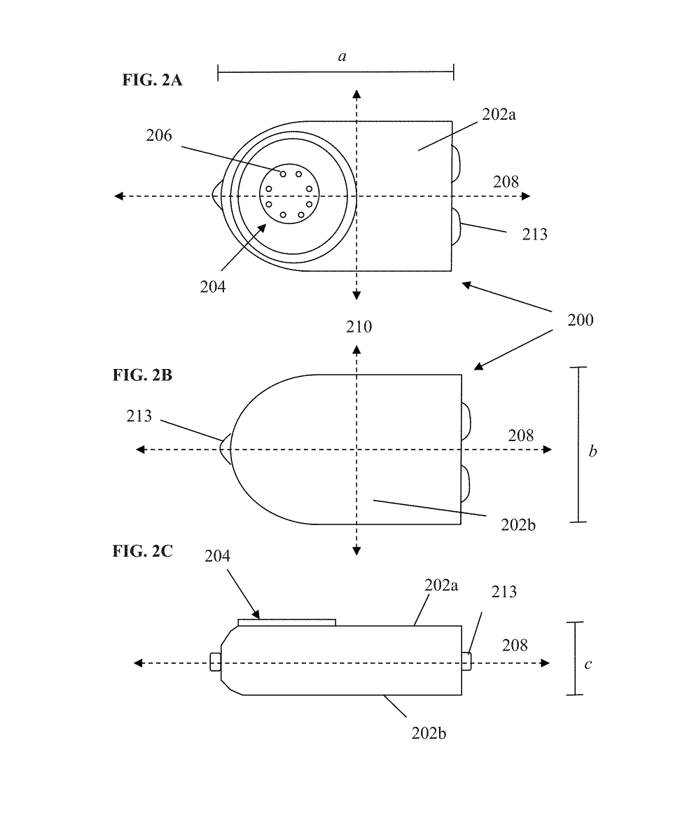

Referring now to FIGS. 2-2C, one exemplary embodiment of a sensor apparatus useful with various aspects of the present disclosure is shown and described.

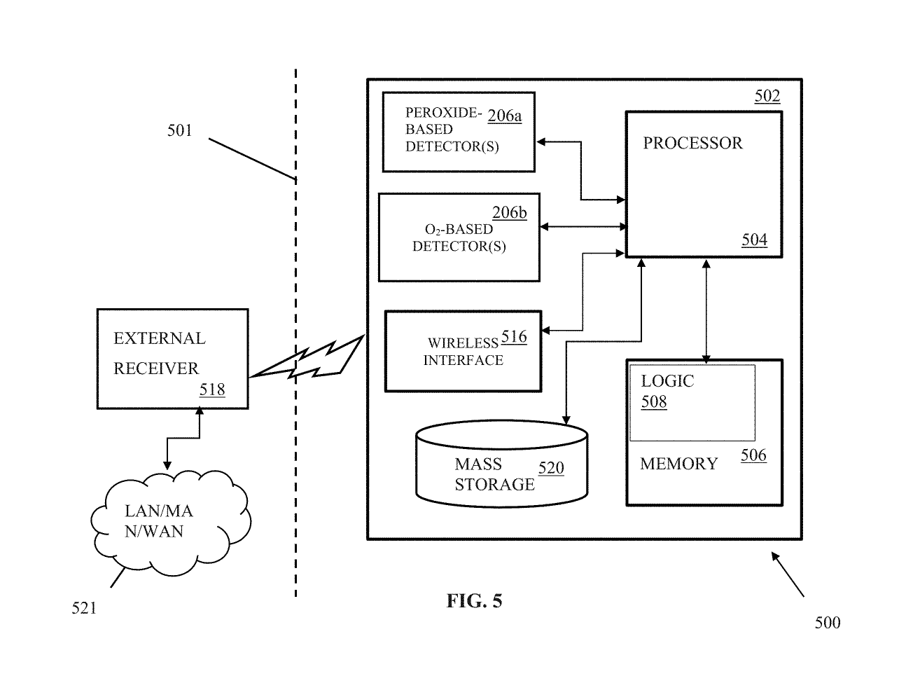

As shown in FIGS. 2-2C, the exemplary sensor apparatus 200 comprises a somewhat planar housing structure 202 with a sensing region 204 disposed on one side thereof (i.e., a top face 202a). As described in greater detail below with respect to FIGS. 4-5, the exemplary substantially planar shape of the housing 202 provides mechanical stability for the sensor apparatus 200 after implantation, thereby helping to preserve the orientation of the apparatus 200 and mitigate any tissue response induced by movement of the apparatus while implanted. Notwithstanding, the present disclosure contemplates sensor apparatus of shapes and/or sizes other than that of the exemplary apparatus 200.

The sensor apparatus of FIGS. 2-2C further includes a plurality of individual sensor elements 206 with their active surfaces disposed substantially within the sensing region 204 on the top face 202a of the apparatus housing. In the exemplary embodiment (i.e., a both hydrogen peroxide-based and oxygen-based multi-element glucose sensor), the eight (8) sensing elements 206 are grouped into four pairs, one element of each pair an active, working, or “primary” sensor associated with an enzyme matrix, and the other a reference or “secondary” sensor (e.g., a “primary” hydrogen peroxide detector element paired with a “secondary” hydrogen peroxide-based detector element, a “primary” oxygen detector element paired with a “secondary” oxygen-based detector element, etc.). In one implementation, the reference or secondary detector for the peroxide-based enzyme electrode is essentially an enzyme-free peroxide detector. This reference or secondary detector is configured to give a non-specific background signal that can be subtracted from the signal of the enzyme-modulated (peroxide) detector, although it will be appreciated that other approaches may be used consistent with the present disclosure.

Further, at least a portion of the four pairs are hydrogen peroxide-based detector elements (i.e., a first detector element type), while the remaining of the pairs are oxygen-based detector elements (i.e., a second detector element type). In the example of FIG. 2A, two (2) of the sensor pairs are hydrogen peroxide-based sensors 206a and the other two (2) of the sensor pairs are oxygen-based sensors 206b.

Exemplary implementations of the sensing elements and their supporting circuitry and components are described in, inter alia, U.S. Pat. No. 7,248,912, previously incorporated herein, U.S. Pat. No. 9,247,901 to Kamath et al. issued Feb. 2, 2016 and entitled “Systems and Methods for Replacing Signal Artifacts in a Glucose Sensor Data Stream”, and U.S. Pat. No. 9,451,908 to Kamath, et al. issued Sep. 27, 2016 and entitled “Analyte Sensor”, each of which is herein incorporated by reference in its entirety. It will be appreciated, however, that the type and operation of the sensor apparatus may vary; i.e., other types of sensor elements/sensor apparatus, configurations, and signal processing techniques thereof may be used consistent with the various aspects of the present disclosure, including, for example, signal processing techniques based on various combinations of signals from individual elements in the otherwise spatially-defined sensing elements pairs.

In the exemplary implementation illustrated in FIGS. 2-2C, the sensor apparatus includes a sensing region 204 which facilitates some degree of “interlock” of the surrounding tissue (and any subsequent tissue response generated by the host) so as to ensure direct and sustained contact between the sensing region 204 and the blood vessels of the surrounding tissue during the entire term of implantation (as well as advantageously maintaining contact between the sensing region 204 and the same tissue; i.e., without significant relative motion between the two). See, e.g., U.S. patent application Ser. No. 15/197,104 filed Jun. 29, 2016 previously incorporated herein for exemplary apparatus and techniques for such sensor apparatus interlock.

The sensor apparatus 200 also includes in the exemplary embodiment a wireless radio frequency transmitter (or transceiver, depending if signals are intended to be received by the apparatus), described in greater detail with respect to FIGS. 5-10 below. As described in the aforementioned documents incorporated herein, the transmitter/transceiver may be configured to transmit modulated radio frequency signals to an external receiver/transceiver, such as a dedicated receiver device, or alternatively a properly equipped consumer electronic device such as a smartphone or tablet computer. Moreover, the sensor apparatus 200 may be configured to transmit signals to (whether in conjunction with the aforementioned external receiver, or in the alternative) an at least partly implanted or in vivo receiving device, such as an implanted pump or other medication or substance delivery system (e.g., an insulin pump or dispensing apparatus), embedded “logging” device, or other. It is also appreciated that other forms of wireless communication may be used for such applications, including for example inductive (electromagnetic induction) based systems, or even those based on capacitance or electric fields, optical (e.g., infrared) systems where a sufficiently clear path of transmission and reception exists, such as two devices in immediately adjacent disposition, or even ultrasonic systems where the two devices are sufficiently close and connected by sound-conductive media such as body tissues or fluids (or a purposely interposed non-body element).

The sensor apparatus of FIGS. 2-2C also includes a plurality (three in this instance) of tabs or anchor apparatus 213 disposed substantially peripheral on the apparatus housing. These anchor apparatus provide the implanting surgeon with the opportunity to anchor the apparatus to the anatomy of the living subject, so as to frustrate translation and/or rotation of the sensor apparatus 200 within the subject immediately after implantation but before any tissue response (e.g., FBR) of the subject has a chance to immobilize (such as via interlock with the sensing region of the apparatus. See e.g., U.S. patent application Ser. No. 14/982,346 filed Dec. 29, 2015 and entitled “Implantable Sensor Apparatus and Methods” previously incorporated herein, for additional details and considerations regarding the aforementioned anchor apparatus 213 (which may include, for example features to receive sutures (dissolvable or otherwise), tissue ingrowth structures, and/or the like).

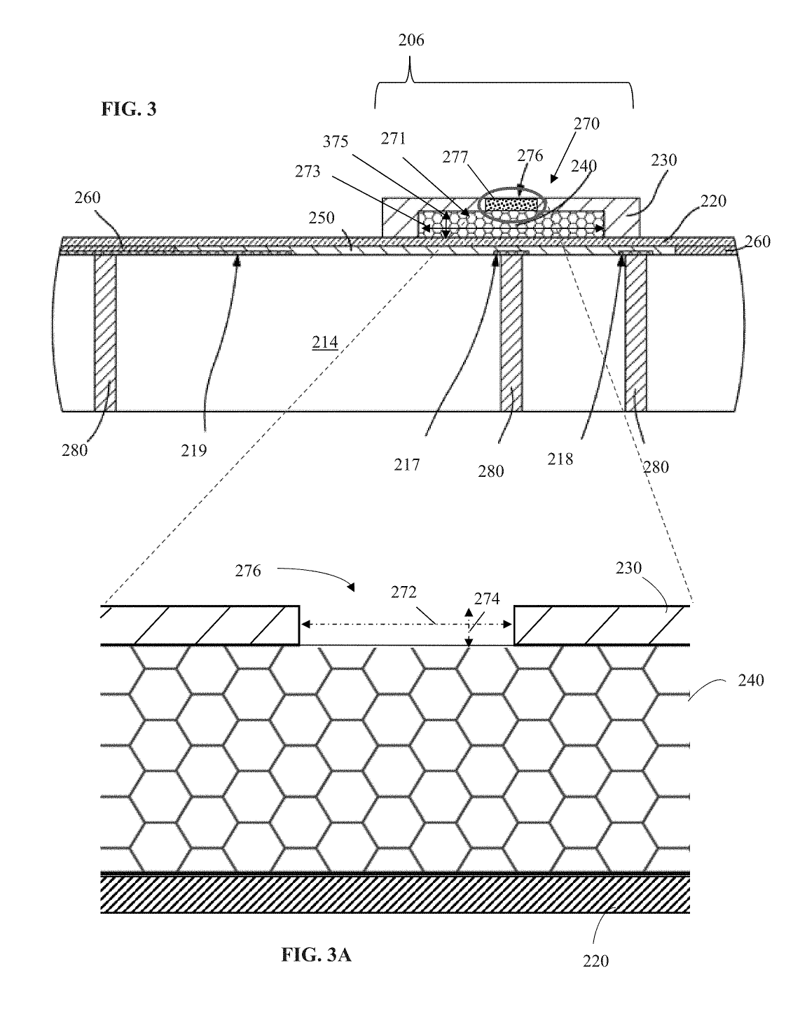

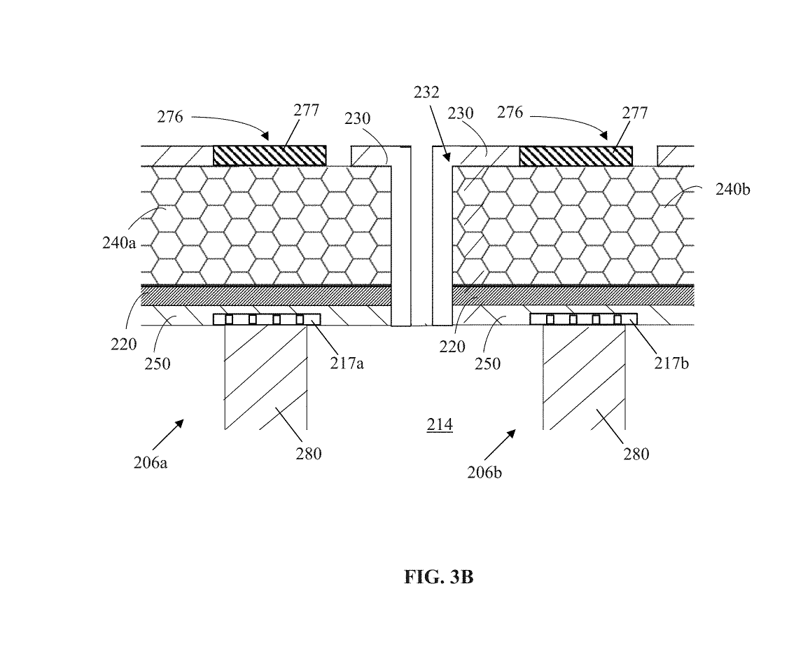

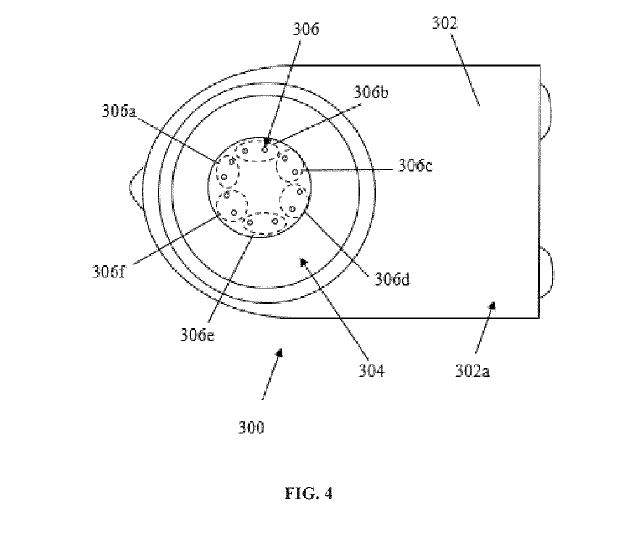

As shown in FIG. 3, an exemplary individual detector element 206 according to the present disclosure is shown associated with detector substrate 214 (e.g. ceramic substrate), and generally comprises a plurality of membranes and/or layers, including e.g., the insulating layer 260, and electrolyte layer 250, an enzymatic gel matrix 240 (e.g., a glucose oxidase embedded matrix, a catalase and glucose oxidase embedded matrix, etc.), an inner membrane 220, an exterior membrane shell 230, and a non-enzymatic membrane 277. Such membranes and layers are associated with the structure of individual detector elements, although certain membrane layers can be disposed in a continuous fashion across the entire detector array surface or portions thereof that include multiple detectors, such as for economies of scale (e.g., when multiple detectors are fabricated simultaneously), or for maintaining consistency between the individual detector elements by virtue of making their constituent components as identical as possible. Further, such membranes and layers can be diffusionally isolated to the associated structure of an individual detector element and/or individual detector element types to maintain proper function of the sensor apparatus. In one exemplary embodiment, any given hydrogen peroxide-based sensor element is diffusionally isolated from any given oxygen-based sensor element. For example, a glucose oxidase embedded gel matrix associated with a hydrogen peroxide-based sensor element is diffusionally isolated from a glucose oxidase and catalase embedded gel matrix (associated with an oxygen-based sensor element) to ensure that digestion of hydrogen peroxide occurs at the hydrogen peroxide-based sensor element surface (rather than via catalase enzymatic digestion). In another example, a sensor element is diffusionally isolated to prevent mass transport of analyte constituents and/or byproducts to sensor elements of a different type.

Generally, the thickness of each of the membranes disclosed herein is not particularly limited, as long as the desired permeability properties are achieved. However, particular requirements for sensor response time, glucose concentration detection range, and/or reduction of antibody response (e.g., FBR), may impose limits on the allowable membrane thickness. Membrane thickness can be, for example, about 1 micron to about 1000 microns, or more particularly, about 10 microns to about 500 microns, or more particularly about 25 microns to about 250 microns in certain applications. Very thin membrane layers, particularly those less than about 10 microns, may require mechanical support to be provided in the form of a backing membrane, which may be a porous, relatively inert structure. U.S. Pat. No. 7,336,984 and entitled “Membrane and Electrode Structure for Implantable Sensor,” previously incorporated herein, describes exemplary membrane apparatus, thickness values, and computerized modeling techniques useful with the various aspects of the present disclosure, although it will be recognized that other techniques, apparatus, and methods for membrane configuration may be used consistent with the present disclosure.

As shown in FIGS. 3 and 3A, the detector elements 206 each further comprise a working electrode 217 (i.e., “primary” electrode) in operative contact (by means of the electrolyte layer 250) with a counter electrode 219 and a reference electrode 218, and their associated feedthroughs 280 (details of the exemplary feedthroughs 280 are described in U.S. Pat. No. 8,763,245 to Lucisano et al. entitled “Hermetic feedthrough assembly for ceramic body,” previously incorporated by reference herein).

In one example, the working electrode 217 (such as e.g., the working electrode of one of detector elements 206a) comprises a hydrogen peroxide-detecting catalytic surface producing a glucose-modulated, oxygen-dependent current (discussed infra), reference electrode 218 comprises an electrochemical potential reference contact to electrolyte layer 250, and counter electrode 219 is operably connected by means of electrolyte layer 250 to the working electrode 217 and reference electrode 218. An electrical potentiostat circuit (not shown) is coupled to the electrodes 217, 218, and 219 to maintain a fixed potential between the working and reference electrode by passing current between the working and counter electrodes while preferably maintaining the reference electrode at high impedance. Such potentiostat circuitry is well known in the art (see for example, U.S. Pat. Nos. 9,247,901 and 9,451,908, previously incorporated herein, and U.S. Pat. No. 4,703,756 to Gough et al. entitled “Complete Glucose Monitoring System with an Implantable, Telemetered Sensor Module,” incorporated herein by reference in its entirety).

The peroxide-based detector elements of the exemplary sensor apparatus of the present embodiment utilize a “hydrogen peroxide-sensing differential measurement,” by comparison of the glucose-dependent hydrogen peroxide detector signal (i.e., from the primary or enzyme-containing sensor elements) to the background hydrogen peroxide detector signal (i.e., from the secondary non-enzyme-containing sensor elements) that produces, upon further signal processing, a substantially continuous real-time blood glucose concentration measurement. It will be appreciated, however, that the methods and apparatus described herein are in no way limited to such “differential” schemes.

In one variant, the enzyme-embedded membrane includes embedded glucose oxidase (GOX) enzymes and the sensor elements are configured for detection of glucose based on the following one-step chemical reaction catalyzed by GOX as described in Wong et al. (Appl Microbiol Biotechnol 78:6, 927-938 (2008))

glucose+O₂→gluconolactone+H₂O₂

A change in hydrogen peroxide concentration can be monitored to determine glucose concentration as the enzyme-catalyzed consumption of glucose molecules is proportional to the production of hydrogen peroxide molecules. Specifically, the hydrogen peroxide produced from the GOX reaction further reacts (i.e., decomposes) at the surface of the working electrode 217 to produce two protons (2H⁺), two electrons (2e⁻), and one oxygen molecule (O₂). Thus, the decomposition of hydrogen peroxide at the working electrode surface produces an electrical current detectable by the working electrode 217. Oxidation of hydrogen peroxide by the working electrode is balanced by reduction of ambient oxygen, enzyme generated hydrogen peroxide, or other reducible species at the counter electrode 219.

In one implementation, the enzyme (GOX in an excess concentration) is immobilized within a gel matrix that is crosslinked for mechanical and chemical stability, and is in operative contact with working electrodes of each of the sensor elements 206a, which are configured to electrochemically sense hydrogen peroxide. Glucose and ambient oxygen diffuse into the gel matrix and encounter the enzyme, the above reactions occur, and decomposition of hydrogen peroxide at the electrode surface is detected by the electrode. In embodiments based on “hydrogen peroxide-sensing differential measurement” (i.e., comparison of an active sensor reading to a reference sensor reading), after comparison of the active hydrogen peroxide concentration reading with the background hydrogen peroxide detector reading, the difference is related to glucose concentration. Thus, hydrogen peroxide produced in the initial GOX catalyzed reaction is digested to oxygen and water (and electrons) via the subsequent decomposition of hydrogen peroxide at the sensor surface, and thereby glucose concentration may be determined via detection of such hydrogen peroxide.

In another example, the working electrode 217 (such as e.g., the working electrode of one of detector elements 206b) comprises an oxygen-detecting catalytic surface producing a glucose-modulated, oxygen-dependent current (discussed infra), reference electrode 218 comprises an electrochemical potential reference contact to electrolyte layer 250, and counter electrode 219 is operably connected by means of electrolyte layer 250 to the working electrode 217 and reference electrode 218. An electrical potentiostat circuit (not shown) is coupled to the electrodes 217, 218, and 219 to maintain a fixed potential between the working and reference electrode by passing current between the working and counter electrodes while preferably maintaining the reference electrode at high impedance. Such potentiostat circuitry is well known in the art (see for example, U.S. Pat. Nos. 9,247,901 and 9,451,908, and U.S. Pat. No. 4,703,756 to Gough et al. each incorporated herein by reference in its entirety).

The O₂-based detectors of the exemplary sensor apparatus of the present embodiment utilize an “oxygen-sensing differential measurement,” by comparison of the glucose-dependent oxygen signal (i.e., from the primary or enzyme-containing sensor elements) to the background oxygen signal (i.e., from the secondary non-enzyme-containing sensor elements) that produces, upon further signal processing, a continuous real-time blood glucose concentration measurement. It will be appreciated, however, that the methods and apparatus described herein are in no way limited to such “differential” schemes.

In one variant, the enzyme-embedded membrane includes embedded glucose oxidase (GOX) and catalase enzymes and the sensor elements are configured for detection of glucose based on the following two-step chemical reaction catalyzed by GOX and catalase as described in Armour et al. (Diabetes 39, 1519-1526 (1990)):

glucose+O₂→gluconate+H₂O₂

H₂O₂→1/2O₂+H₂O

resulting in the overall enzyme reaction (when catalase is present):

glucose+1/2O₂→gluconate

In one specific implementation, the two enzyme types (GOX and catalase, each in an excess concentration) are immobilized within a gel matrix that is crosslinked for mechanical and chemical stability, and is in operative contact with electrodes of each of the sensor elements, which are configured to electrochemically sense oxygen. Glucose and ambient oxygen diffuse into the gel matrix and encounter the enzymes, the above reactions occur, and oxygen that is not consumed in the process is detected by the electrodes. In embodiments based on “oxygen-sensing differential measurement” (i.e., comparison of an active sensor reading to a reference sensor reading), after comparison of the enzyme-modulated oxygen concentration reading with the background oxygen concentration reading, the difference is related to glucose concentration. Thus, hydrogen peroxide produced in the initial GOX catalyzed reaction is digested to oxygen and water via the subsequent catalase catalyzed reaction, and glucose concentration may be determined via detection of oxygen.

As can be seen in FIGS. 2 and 2A, the sensor pairs are radially arranged and may be substantially evenly spaced apart. An active sensor and a reference sensor are adjacent pairs of sensor elements such that the arrangement will allow each active sensor in the pair to remain within the same relatively homogenous region of the otherwise heterogeneous tissue in which the device is implanted.

The electrolyte layer 250 comprises, in the illustrated embodiment, a layer of hydrophilic electrolyte material which is in direct contact with the working electrode(s) 217, reference electrode(s) 218 and counter electrode(s) 219. In various implementations, materials for constructing the hydrophilic electrolyte layer 250 include salt-containing gels comprising polyacrylamide, poly(ethylene oxide), polyhydroxyethylmethacrylate and its derivatives, and other hydrophilic polymers and copolymers, in both crosslinked and non-crosslinked form. Various other construction details of the exemplary electrolyte layer 250 are described in U.S. Patent Application Publication No. 2013/0197332 filed Jul. 26, 2012 entitled “Tissue Implantable Sensor With Hermetically Sealed Housing,” incorporated by reference herein in its entirety.

In an exemplary embodiment, the enzymatic material 240 comprises a crosslinked gel of hydrophilic material including enzymes (e.g., glucose oxidase or glucose oxidase and catalase) immobilized within the gel matrix, including a buffer agent and small quantities of a chemical crosslinking agent. The hydrophilic material 240 is permeable to both a large molecule component (e.g. glucose) and small molecule components (e.g. hydrogen peroxide and oxygen). In various embodiments, specific materials useful for preparing the enzymatic material 240, include, in addition to an enzyme component, polyacrylamide gels, glutaraldehyde-crosslinked collagen or albumin, polyhydroxy ethylmethacrylate and its derivatives, and other hydrophilic polymers and copolymers, in combination with the desired enzyme or enzymes. The enzymatic material 240 can similarly be constructed by crosslinking glucose oxidase or other enzymes with chemical crosslinking reagents, without incorporating additional polymers.

The enzymatic material 240 is in operative contact with the working electrode 217 through the inner membrane 220 and the electrolyte layer 250 to allow for the electrochemical detection of analyte constituents and/or byproducts (e.g., oxygen, hydrogen peroxide, etc.) at the working electrode(s) 217 modulated by the chemical catalysis reactions discussed above. To that end, as glucose and ambient oxygen diffuse into the enzymatic material 240 from the outer (non-enzymatic) membrane 277, they encounter the resident enzyme(s) (e.g., glucose oxidase or glucose oxidase and catalase) and react therewith. In the example of sensor elements 206a, the hydrogen peroxide produced in the catalytic reaction diffuses through the inner membrane 220 and is detected at the working electrode 217 to yield a glucose-dependent hydrogen peroxide signal. In the example of sensor elements 206b, the oxygen that is not consumed in the reaction(s) diffuses through the inner membrane 220 and is detected at the working electrode 217 to yield a glucose-dependent oxygen signal.

A selective permeability material is utilized for inner membrane 220, which is shown in FIG. 3 as being disposed over the electrolyte layer 250. The material is impermeable to the larger or less soluble molecule component (e.g. glucose) but permeable to the smaller or more soluble molecule components (e.g. hydrogen peroxide and/or oxygen). In various embodiments, materials useful for preparing selective permeability layers, including inner membrane 220, as well as membrane shell 230, include organosilicon polymers, such as polydimethylsiloxane (PDMS) and derivatives thereof, polymers of tetrafluoroethylene, ethylene tetrafluoroethylene, or fluorochloro analogs alone or as copolymers with ethylene or propylene, polyethylene, polypropylene, cellulose acetate, polyurethanes, and other oxygen-permeable and/or hydrogen peroxide-permeable polymeric materials, chosen based on their selectivity for the solute of interest. It is recognized that: (i) selectively permeable materials may be sufficiently permeable to pass both oxygen and peroxide molecules, and hence a “dual-purpose” material may be used in some configurations; and (ii) permeable materials that are permeable to one species or molecule (e.g., oxygen molecules) may not be sufficiently permeable to pass another species or molecule (e.g., peroxide molecules), or vice-versa, and hence two or more materials and/or regions may be used in other configurations, such that both permeability of oxygen and peroxide are addressed.

In one example, the inner membrane 220 is associated with a single detector set or detector type in order to prevent drift due to diffusion of oxygen and/or hydrogen peroxide through the inner membrane to adjacent detection regions (i.e., regions of detection for other detector sets or detector types). The inner membrane 220 can alternatively be a continuous layer across the entire detector array surface, and thus be a single common layer utilized by all detectors in the detector array. It is noted that the inner membrane 220, inter alia, protects the working electrode 217, reference electrode 218 and counter electrode 219 from drift in sensitivity due to contact with certain confounding phenomena (e.g. electrode “poisoning”), but the working electrode 217 will nonetheless be arranged sufficiently close to the enzymatic material to enable detection of analyte constituents and/or byproducts (e.g., hydrogen peroxide or oxygen) levels therein.

The (hydrophobic) outer membrane shell 230 is disposed over at least a portion of the enzymatic material 240 (forming a cavity 271 within which the material 240 is contained), and is further configured to include an aperture within a “spout” region 270 (discussed infra). It is contemplated that the inner membrane 220 and the membrane shell 230 can be coextensive and therefore be disposed as one continuous membrane layer in which outer membrane shell 230 and inner membrane 220 are of the same uniform thickness of membrane across the individual detector and array, although it will be appreciated that other thicknesses and configurations may be used as well, including configurations wherein the membrane shell 230 is separately provided and adhesively bonded to the inner membrane 220.

As discussed elsewhere herein, in order to ensure accurate measurement of glucose-dependent hydrogen peroxide signals and glucose-dependent oxygen signals, the (peroxide-based) sensor elements 206a are diffusionally isolated from the (oxygen-based) sensor elements 206b. Accordingly, hydrogen peroxide produced in the glucose oxidase catalyzed reaction associated with the sensor elements 206a subsequently diffuses through the inner membrane 220 and the electrolyte layer 250 to the working electrode 217a and reacts at the electrode surface for detection (rather than being enzymatically digested via catalase or diffusing toward sensor element 206b). Further, unconsumed oxygen (i.e., oxygen unconsumed in the glucose oxidase and catalase catalyzed reactions associated with the sensor elements 206b) subsequently diffuses through the inner membrane 220 and the electrolyte layer 250 to the working electrode 217b for detection (rather than diffusing toward sensor element 206a). Furthermore, oxygen produced in the decomposition of hydrogen peroxide at the surface of the working electrode 217a of sensor element 206a is prevented from diffusing toward sensor element 206b.

One exemplary embodiment for diffusional isolation of adjacent sensor elements (such as e.g., sensor elements 206a and 206b) is shown in FIG. 3B. In this embodiment, the outer membrane shells 230 are partially extended over the outer surfaces of enzymatic gel matrices 240a and 240b (i.e., a glucose oxidase embedded matrix and a catalase and glucose oxidase embedded matrix, respectively) and form adjacent apertures 276 (having non-enzymatic membranes 277 disposed therein). The outer membrane shells 230 are extended from an outer surface of enzyme gel matrices 204a and 204b to the detector substrate 214 (through the inner membranes 220 and the electrolyte layers 250) in between the adjacent sensor elements 206a and 206b. Thus, the outer membrane shells 230 and a gap or space 232 is disposed between the outer membrane shells 230 prevent diffusion of molecules (through the enzymatic gel matrices 240a and 240b, the inner membranes 220, and the electrolyte layers 250) to adjacent sensor elements 206a and 206b. It will be appreciated that the example shown in FIG. 3B is just one example for diffusional isolation and other configurations are contemplated (e.g., a contiguous outer membrane shell disposed between the enzyme gel matrices 240a and 240b lacking a space or gap, etc.).

Returning to FIG. 3, the inner membrane 220 and the membrane shell 230 are disposed in a manner that creates discrete three-dimensional regions having different thicknesses on the detector substrate 214 (see also FIG. 3B), which can be utilized to create tissue anti-migration elements used to achieve stability of location, prevention of device migration away from its original implant location, and prevention of local tissue slippage in the vicinity of the detector element 206. Alternatively, the hydrophobic component may be dispersed as small domains in a continuous phase of the hydrophilic material. Various other construction details of the hydrophobic component dispersed as small domains in a continuous phase of hydrophilic material are described in U.S. Pat. Nos. 4,484,987 and 4,890,620, each incorporated herein by reference in its entirety.

As shown in FIGS. 3-3A, a single spout region 270 of the detector element 106 forms a small opening or aperture 276 through the membrane shell 230 to constrain the available surface area of hydrophilic enzymatic material 240 exposed for diffusionally accepting the analyte and/or co-reactant(s) (e.g. glucose and oxygen) from solution. Alternatively, it is contemplated that one or more spout regions (and or apertures within a spout region) can exist per detector element (i.e., per primary detector element). Notably, co-reactant oxygen can enter into the enzymatic material 240 by means of diffusion through the wall of the membrane shell 230. Such additional surface area thus afforded for oxygen diffusion relative to the limited area of the aperture 276 (by which the primary analyte, e.g. glucose, can enter) helps prevent the enzyme reaction rate from becoming prematurely limited by the supply of oxygen, which is of particular value when, inter alia, the oxygen concentration in the environment surrounding the sensor is lower than that of the primary analyte (e.g. glucose).

The shape and dimension of spout region 270 aids in controlling the rate of entry of the analyte and/or co-reactant(s) (e.g. glucose and oxygen) into enzymatic material 240, and thus impacts the effective operational permeability ratio of the enzymatic material 240. The permeability ratio can be expressed as the maximum detectable ratio of glucose to oxygen concentration of an enzymatic glucose sensor. In the example of detector elements 206a, the permeability ratio is the maximum detectable ratio of glucose concentration to oxygen concentration where the sensing element measurement (of glucose) is based on the detection of hydrogen peroxide produced by the enzymatic reaction (and digested at the sensor element surface), after taking into account the effects of external mass transfer conditions and the enzyme reaction stoichiometry.

In the example of detector elements 206b, the permeability ratio is the maximum detectable ratio of glucose concentration to oxygen concentration where the sensing element measurement (of glucose) is based on the detection of oxygen unconsumed by the enzymatic reactions, after taking into account the effects of external mass transfer conditions and the enzyme reaction stoichiometry. Detailed discussions of the relationship between membrane permeability ratio and the maximum detectable ratio of glucose to oxygen concentration of oxygen-detecting, enzymatic, membrane-based sensors are provided in “Model of a Two-Substrate Enzyme Electrode for Glucose,” J. K. Leypoldt and D. A. Gough, Analytical Chemistry, 56, 2896 (1984) and “Diffusion and the Limiting Substrate in Two-Substrate Immobilized Enzyme Systems,” J. K. Leypoldt and D. A. Gough, Biotechnology and Bioengineering, XXIV, 2705 (1982), incorporated herein by reference. The membranes of the exemplary detector element described herein are characterized by a permeability ratio oxygen to glucose of about 200 to about 1 in units of (mg/dl glucose) per (mmHg oxygen). Note that while this measure of permeability ratio utilizes units of a glucose concentration to an oxygen concentration, it is nevertheless a measure of the ratio of oxygen to glucose permeability of the membrane.

The exemplary spout 270 is formed as a void in the hydrophobic material of the membrane shell 230 (e.g., silicone rubber) and advantageously includes a non-enzymatic outer layer or membrane 277 to, inter alia, prevent direct contact of the immobilized enzymes in the enzymatic material 240 with the surrounding tissue, thereby eliminating and/or reducing antibody response, encapsulation, and/or other deleterious factors. In exemplary embodiments, the non-enzymatic membrane 277 is further constructed (i.e., with a substantially planar crosslinked biocompatible matrix possessing pores substantially smaller than those required to accommodate blood vessel ingrowth, but large enough to accommodate diffusion of the analyte(s) and/or co-reactant(s)) so as to frustrate or mitigate blood vessel formation therein. (Suitable pores include those with an effective diameter ranging from approximately 10 angstroms up to approximately 10 microns.) Herein lies a salient feature of the sensor element of the exemplary embodiment; i.e., the combination of (i) an enzyme-free biocompatible outer membrane 277, (ii) maintenance of the spout region substantially free of enzyme material during manufacture (see discussion of manufacturing methods below), (iii) use of a non-porous crosslinked structure for the membrane 277, and (iv) use of a biocompatible material (e.g., silicone rubber) for the outer membrane shell 230, dramatically reduces the level of FBR of the host while the device is implanted, thereby allowing for both longer implantation (due to, inter alia, the reduced level of FBR not interfering with sensor operation) and easier explants of the device, as compared to e.g., peroxide-based sensors without such features. The inner hydrophobic membrane 220 further provides additional insulation of the host tissue in the region of the detector 106 against any electrical potentials which may be present with in the sensor element, thereby further aiding in mitigating FBR. In various implementations, materials for constructing the membrane layer 277 include gels comprising proteins such as albumin and collagen, as well as non-proteinaceous polymers such as polyacrylamide, poly(ethylene oxide) poly(hydroxyethylmethacrylate) and its derivatives, and other hydrophilic polymers and copolymers, in both crosslinked and non-crosslinked form.

The spout aperture diameter 272 in part controls the effective operational membrane permeability ratio. In the exemplary embodiment, the aperture diameter is correlated to the range of concentration of the target analyte (e.g., glucose) that can be detected by the detector element. For both detector element types 206a and 206b (i.e., hydrogen peroxide-based glucose detectors and oxygen-based glucose detectors, respectively), larger aperture diameter corresponds to a lower permeability ratio of oxygen to glucose, and hence a greater sensitivity to a given concentration of glucose within the tissue proximate the aperture (and therefore a lower minimum concentration that can be accurately detected). However, with the larger aperture, the detector will “saturate” with respect to the primary analyte more rapidly at a given oxygen concentration, and hence the upper bound of detection is similarly reduced. Conversely, a smaller diameter aperture corresponds to an increased permeability ratio, and hence a higher minimum effective sensitivity (and corresponding higher maximum detectable concentration before saturation is reached).

It is also appreciated that: (i) in various embodiments, the aperture 276 of the spout region 270 may be virtually any geometric shape so long as a desired permeability ratio is achieved, such as, for example, round, oval, elliptical, rectangular, triangular, star shaped, square, polygonal, and the like (see FIG. 4), or even irregular, although round (circular) apertures are generally preferred because such shapes are more amenable to manufacturing; and (ii) diameter is straightforwardly related to area, and the present disclosure contemplates that area may be a useful measure of “spout size” as it relates to adjusting the operational characteristics of the detector element in place of diameter. See U.S. patent application Ser. No. 15/170,571 filed Jun. 1, 2016 previously incorporated herein for exemplary methods and apparatus relating to the spout region of the sensor detector elements 206b, which in an exemplary embodiment of the present disclosure, can be directly extended to use with the peroxide-based detector elements 206a of the apparatus 200 of FIG. 2.

As will be apparent to those skilled in the art, the outer (non-enzymatic) membrane layer 277 can be formed in any number of different ways. In the exemplary embodiments (see FIGS. 3, 3B, and 5A-5D), the non-enzymatic layer 277 is in effect “pour filled” into the aperture 276 of the outer housing membrane 230 atop the (crosslinked) enzymatic membrane matrix 240. However, the present disclosure contemplates other techniques for formation, including for example provision of a pre-formed membrane 277 which is inserted into the aperture in operative contact with the enzyme material 240, or even chemical or other treatments of the upper surface of enzyme material 240, including various de-immunizing treatments. See U.S. patent application Ser. No. 15/170,571 filed Jun. 1, 2016 previously incorporated herein for exemplary methods of forming the detector elements, and manufacturing of the sensor apparatus as a whole.

In all cases, it is required that the outer membrane layer 277 (where used) be sufficiently permeable to analytes to enable correct operation of the detector. In the exemplary embodiment, the outer membrane 277 comprises crosslinked albumin, which exhibits the aforementioned desirable properties of (i) lack of FBR-inducing enzymes, (ii) non-supportive of blood vessel ingrowth, and (iii) permeable to analytes. Notably, the exemplary albumin material used for the membrane layer 277 is biocompatible; in the present context (a tissue-located implant), the term ‘biocompatible’ as applied to the membrane layer 277 indicates that the material itself does not significantly exacerbate the FBR which is otherwise expected to occur with any implant. So, the amount/degree of fibrous tissue formation that results from the FBR (which nonetheless occurs due to natural body processes) is advantageously minimized, compared to what might be obtained with another less-biocompatible material.

Notably, in the exemplary implementation, the hydrophilic albumin of the outer membrane 277 is in direct contact with the (hydrophilic) tissue of the host, thereby advantageously providing a “like-to-like” interface, which also contributes to the stability of the detector elements over time due to, among other things, the aforementioned non-exacerbation of FBR or other host responses.

It is also noted that the exemplary membrane layer 277 described herein, by virtue of its non-exacerbation of FBR in the host (e.g., through use of a biocompatible material such as crosslinked albumin), further results in mitigation of the formation of significant fibrous tissue response, which could otherwise interfere with optimal operation of the sensor detector elements or reduce their accuracy due to, inter alia, reduced blood vessel density in the fibrous tissue. So, in effect, the non-exacerbation of FBR and non-encouragement of blood vessel ingrowth into the membrane layer 277 by the exemplary embodiment herein actually (and somewhat counter-intuitively) stabilizes tissue perfusion and blood glucose delivery to the detector elements, and avoids having to second-guess the largely unpredictable modulation process, especially over longer periods of implantation.

Other biostable polymers suitable as outer (housing) membrane materials include, for example, hydrophilic polyurethanes, silicones, poly(hydroxyethylmethacrylate)s, polyesters, polyalkyl oxides (polyethylene oxide), polyvinyl alcohols, and polyethylene glycols and polyvinyl pyrrolidone. See, inter alia, U.S. Patent Application Publication No. 2013/0197332 previously incorporated herein, for a discussion of other various outer membrane materials.

It is contemplated that in other embodiments, the detector array 104 includes detector elements 106 with different spout (aperture) diameters and/or other physical characteristics (such as those described above) in order to provide detector element arrays having heterogeneous detection ranges. For example, a sensor apparatus can include one detector or set of detectors with larger spout diameters and/or heights, and another detector/set with smaller spout diameters and/or heights. Having multiple detector elements 106 with such different physical characteristics (and hence operating characteristics) is beneficial for any number of reasons, including maintaining a broader desired sensor response range. Further, characteristics of the spout can be optimized for the particular detector element type (e.g., hydrogen peroxide-based glucose detector element, oxygen-based glucose detector element, etc.). See U.S. patent application Ser. No. 15/170,571 filed Jun. 1, 2016 previously incorporated herein, for exemplary methods and apparatus for heterogeneous detector configuration and use. For example, each primary hydrogen peroxide detector element associated with a glucose oxidase embedded membrane is paired with a secondary hydrogen peroxide detector element (i.e., a non-enzyme matrix associated element), and each primary oxygen detector element associated with a glucose oxidase and catalase embedded matrix is paired with a secondary oxygen sensor (i.e., a non-enzyme matrix associated element). In the example of FIG. 2A, three (3) of the sensor pairs are hydrogen peroxide sensors 306a, 306b, 306c and the other three (3) of the sensor pairs are oxygen sensors 306d, 306e, 306f. Each of 306a and 306d are configured to measure glucose level within a first response range, each of 306b and 306e are configured to measure glucose level within a second (at least partly differing) response range, and each of 306c and 306f are configured to measure glucose level within a third range (at least partly different from the first and second ranges).

Moreover, another exemplary embodiment of the sensor apparatus described herein may include either or both of: (i) multiple detector elements with respective “staggered” ranges/rates of detection operating in parallel, and/or (ii) multiple detector elements with respective “staggered” ranges/rates of detection that are selectively switched on/off in response to, e.g., the analyte concentration reaching a prescribed upper or lower threshold, as described in the foregoing patent application Ser. No. 15/170,571.

The present disclosure further contemplates that such thresholds or bounds: (i) can be selected independent of one another; and/or (ii) can be set dynamically while the apparatus 300 is implanted. For example, in one scenario, operational detector elements are continuously or periodically monitored to confirm accuracy, and/or detect any degradation of performance (e.g., due to equipment degradation, progressive FBR affecting that detector element, etc.); when such degradation is detected, affecting say a lower limit of analyte concentration that can be detected, that particular detector element can have its lower threshold adjusted upward, such that handoff to another element capable of more accurately monitoring concentrations in that range.

Alternatively, each of the aforementioned heterogeneous sensor sets 306a-f may simply be operated in parallel, and data generated by each transmitted off-device (e.g., via wireless interface to an external receiver) for subsequent processing of the raw data on the external receiver device or on an external computational platform, such as via application software running on a personal computer or server and configured to identify the most optimal data from each sensor set 306 within the “raw” data generated by that sensor set and transmitted off-device, and utilize the identified optimal data to provide a representation of the measured analyte concentration over the entire range of values encountered, ostensibly with greater accuracy than that provided by a comparable homogenous detection range detector element configuration.

It will be appreciated that the relatively smaller dimensions of the sensor apparatus (as compared to many conventional implant dimensions)—on the order of 40 mm in length (dimension “a” on FIGS. 2A-2C) by 25 mm in width (dimension “b” on FIGS. 2A-2C) by 10 mm in height (dimension “c” on FIGS. 2A-2C)—may reduce the extent of injury (e.g., reduced size of incision, reduced tissue disturbance/removal, etc.) and/or the surface area available for blood/tissue and sensor material interaction, which may in turn reduce intensity and duration of the host wound healing response. It is also envisaged that as circuit integration is increased, and component sizes (e.g., lithium or other batteries) decrease, and further improvements are made, the sensor may increasingly be appreciably miniaturized, thereby further leveraging this factor.

It is also appreciated that some flexibility in component location exists; as such, the present disclosure further contemplates e.g., relocation of certain components within the implanted sensor device 200, 300 such as those associated with signal processing, off-device (i.e., in an external receiver module or other electronic apparatus external to the implanted sensor, such as a user's smartphone or tablet computer, or other implanted or external medical device) so as to further minimize interior sensor device volume/area requirements. For instance, in one such adaptation, electronic components such as antennas and/or circuit boards (e.g., PCBs) can be wholly or partly replaced with so-called “printable” electronics which reside on, e.g., interior components or surfaces of the sensor device 200, 300, such as by using the methods and apparatus described in U.S. Pat. No. 9,325,060 issued Apr. 26, 2016 and entitled “Methods and Apparatus for Conductive Element Deposition and Formation,” which is incorporated herein by reference in its entirety. Other types of space/area-reducing adaptations will be readily recognized by those of ordinary skill in the electronic arts when given the present disclosure.

Returning again to FIGS. 2-2C and 3, the housing 202/302 and sensing region 204, 304 purposely have relatively smooth outer surfaces, which may be comprised of biocompatible materials, thereby limiting reaction of the tissue to the sensor apparatus 200, 300 and allowing long-term implantation. Specifically, by providing a smooth surface over much of the sensor housing and forming the housing (and other externally exposed components) of materials which do not incite tissue response (e.g., titanium), very little if any bonding or attachment of the tissue response to the sensor housing or other such components occurs, even after an extended period of implantation (e.g., 12 months or more). Biocompatible materials may be used at least where any portion of the sensor comes into physical contact with the body. Exemplary biocompatible materials are disclosed in U.S. Patent Publication No. 2013/0197332, previously incorporated herein. A variety of suitable medical grade materials are known in the art which may be utilized to construct the housing; e.g., a metallic material or an alloy such as, but not limited to, bio-inert metals, cobalt-chromium alloys, alloys of cobalt, nickel, chromium and molybdenum, stainless steel, tantalum, tantalum-based alloys, nickel-titanium alloy, platinum, platinum-based alloys such as e.g., platinum-iridium alloy, iridium, gold, titanium, titanium-based alloys, zirconium-based alloys, or combinations thereof. Further, the housing may be constructed from biocompatible ceramic materials, comprising oxides, carbides, borides, nitrides, and silicides of aluminum, zirconium, beryllium, silicon, titanium, yttrium, hafnium, magnesium and zinc.

Furthermore, the housing may also be made from biocompatible, biostable polymers, such as polymers including but not limited to fluorpolymers (e.g., DuPont Teflon® or Tefzel® or the like), epoxy resins, polyetherimides, poly ether ketone, polysulfone, polyphenylsulfone, polypropylene, polycarbonate, poly methyl methacrylate, and others, which may present a smooth and substantially non-adherent surface in certain formulations.

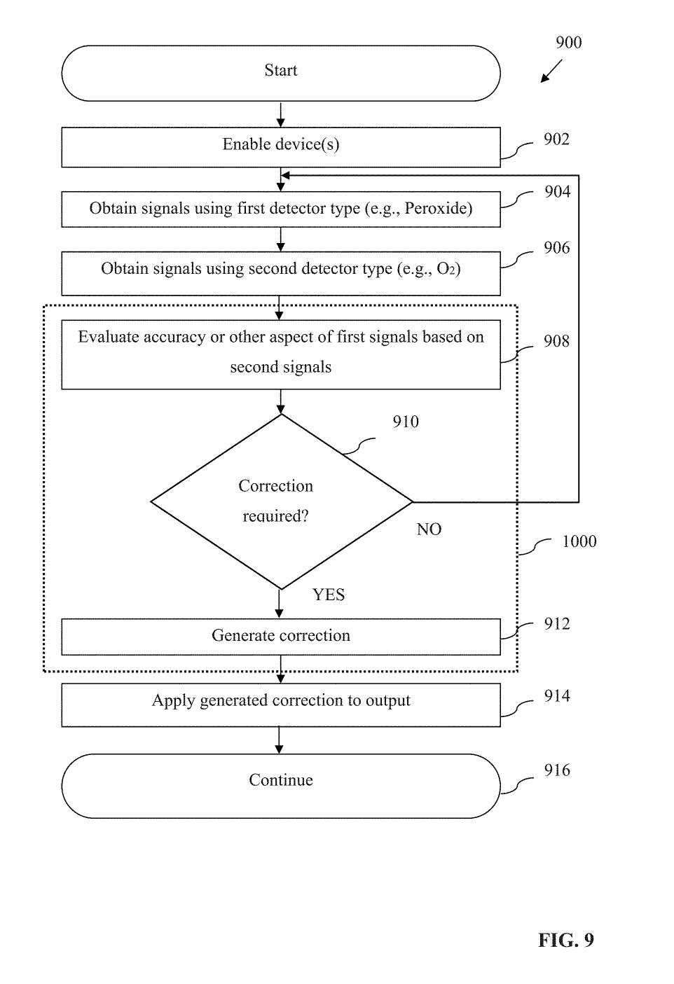

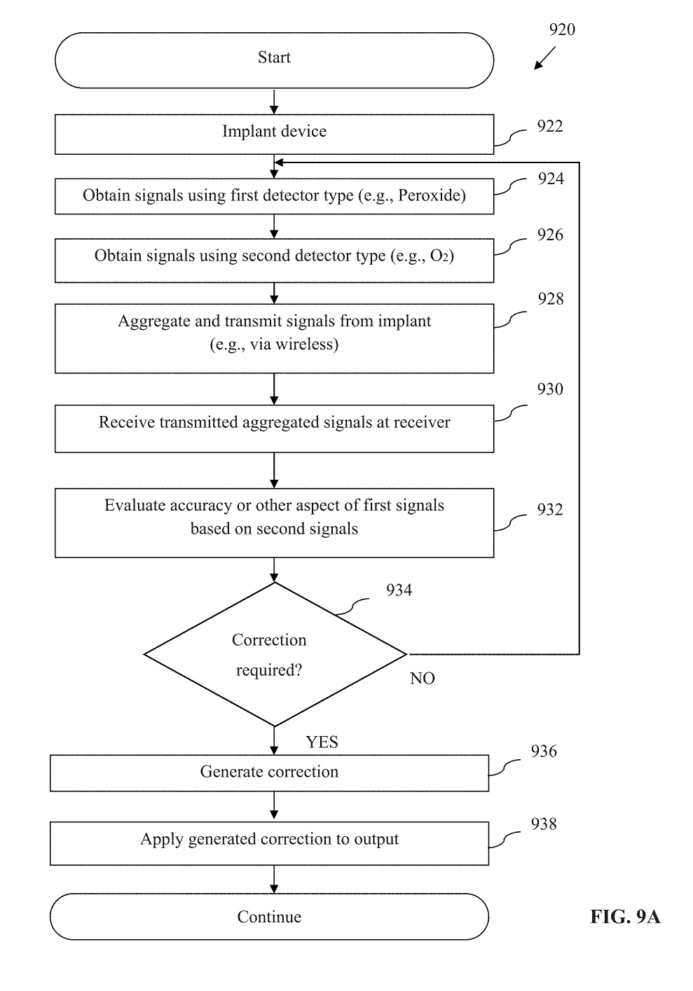

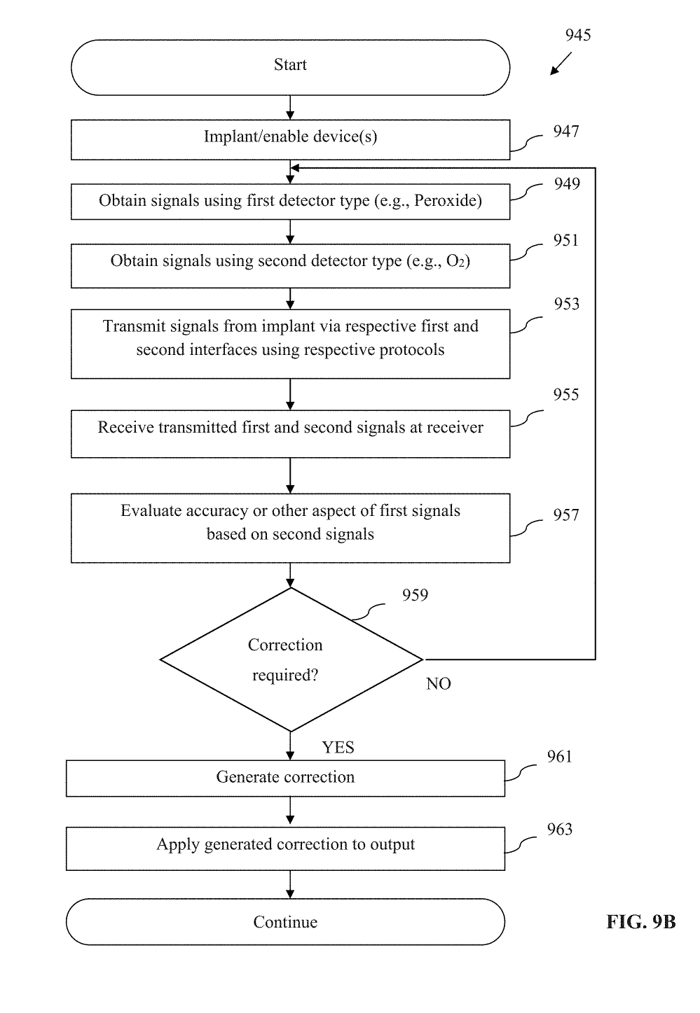

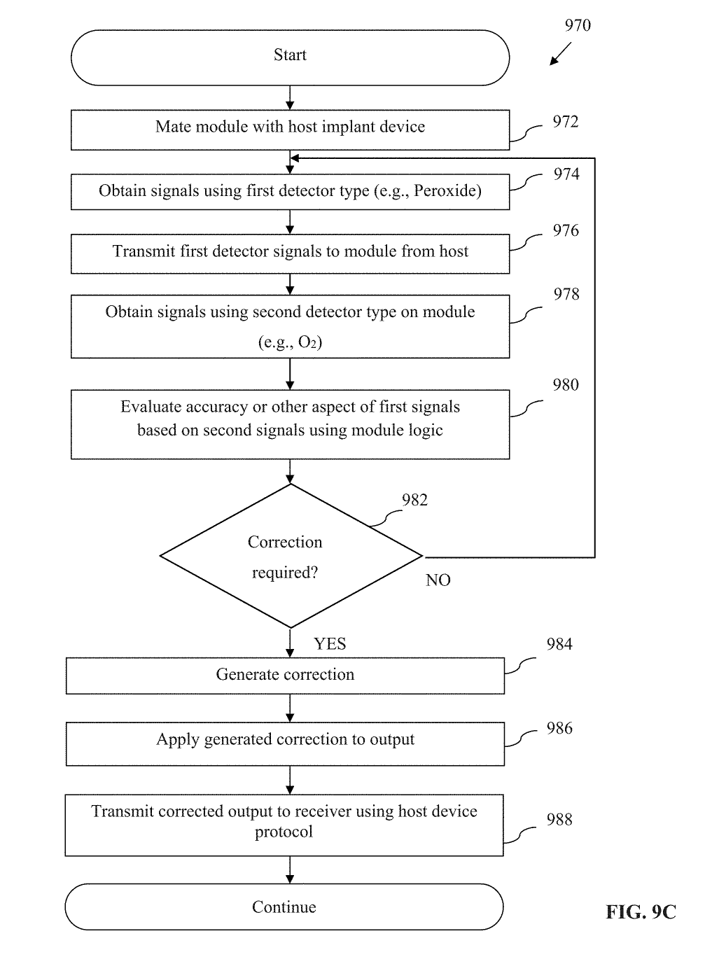

Notably, however, the sensing region 204, 304 of the exemplary sensor apparatus 200, 300 purposely includes some level of texture or relief (albeit with biocompatible materials as well), so as to give any tissue response or encapsulation in that region something to “grab onto” to promote the close contact, interlock, and anti-slip described herein with respect to FIG. 9. Such texture or relief can be provided in one or more ways, including e.g., via a roughened or “bumpy” material texture, and/or one or more prominent or salient features elevated and/or depressed over/within the surrounding portions of the apparatus (i.e., irrespective of texture of the materials). As previously referenced, this arrangement also helps maintain the sensor element active areas (i.e., the outer non-enzymatic membrane 277 and its underlying enzyme in the “primary” sensor elements, and the membrane 277 with non-enzymatic matrix in the “secondary” sensor elements) maintain a substantially constant, direct, and non-variable level of contact with particular blood vessels located in the tissue of that region, so as to maximize the stability and accuracy of the signals generated from each of the sensor elements. Specifically, the absence of relative motion between each individual sensor primary or secondary element and the surrounding tissue and vasculature allows the sensor to receive a substantially constant blood (and hence glucose) supply over time, which translates to a substantially constant rate of glucose diffusion through the outer membrane 277 and into the underlying matrix.

### System Architecture

Referring now to FIGS. 5-7, various embodiments of the sensor apparatus system architecture of the present disclosure are described in detail.

FIG. 5 is a functional block diagram of a first embodiment of an implantable heterogeneous sensing device 502 and associated receiver apparatus 518, according to the disclosure. As shown in FIG. 5, the implantable device 502 (e.g., the apparatus 200 previously described herein) comprises the first detectors 206a (e.g., hydrogen peroxide-based) and the second detectors 206b (e.g., O2-based), a processor apparatus 504, wireless interface 516, program and data memory 506 (e.g., RAM/ROM/PROM), mass storage (e.g., NAND or NOR flash) 520, and logic 508 (e.g., software/firmware operative to be executed on the processor apparatus 504 to provide the desired functionality of the various components). It will be appreciated that while the exemplary embodiment of FIG. 5 (and others) is shown and described with respect to software/firmware 508 (i.e., computer programs configured to be executed on a digital processing apparatus) stored on the memory 506 (the latter which may include, for example, storage locations on the processor apparatus 504, and wireless interface 516), any number of other configurations may be utilized consistent with the present disclosure, including without limitation hardware, programmable gate logic (e.g., FPGA), and application-specific devices (e.g., ASICs), the illustrated embodiments being merely exemplary.

Moreover, while a number of different components are shown, various configurations (with more or less components, including combinations of two or more components) are envisaged. For instance, a mass storage device 520 may be obviated if no on-device mass data storage is desired, or the mass storage may be integrated into the memory device 508, etc.

As shown, the wireless interface 516 is configured to communicate with the external receiver device 518 via a one-way or two-way wireless data protocol and associated modulation scheme suitable for reliable transmission from the implanted device 502 in vivo through any interposed tissue 501 or other substances to the receiver 518 external to the body. The external receiver 518 may also be in data communication with a LAN, WAN, MAN, or other network or device 521 for, e.g., uploading obtained data to a “cloud” entity for later retrieval and/or analysis.

The architecture 500 of FIG. 5 is, in the exemplary embodiment, configured to utilize the signals/data obtained from the two detector types 206a, 206b to generate a calibrated or corrected output that accurately reflects blood glucose level within the host at any given time, whether via on-device 502 processing, off-device (e.g., receiver 518 or other entity in communication therewith) processing, or combinations thereof, as described subsequently herein with respect to FIGS. 9-11.

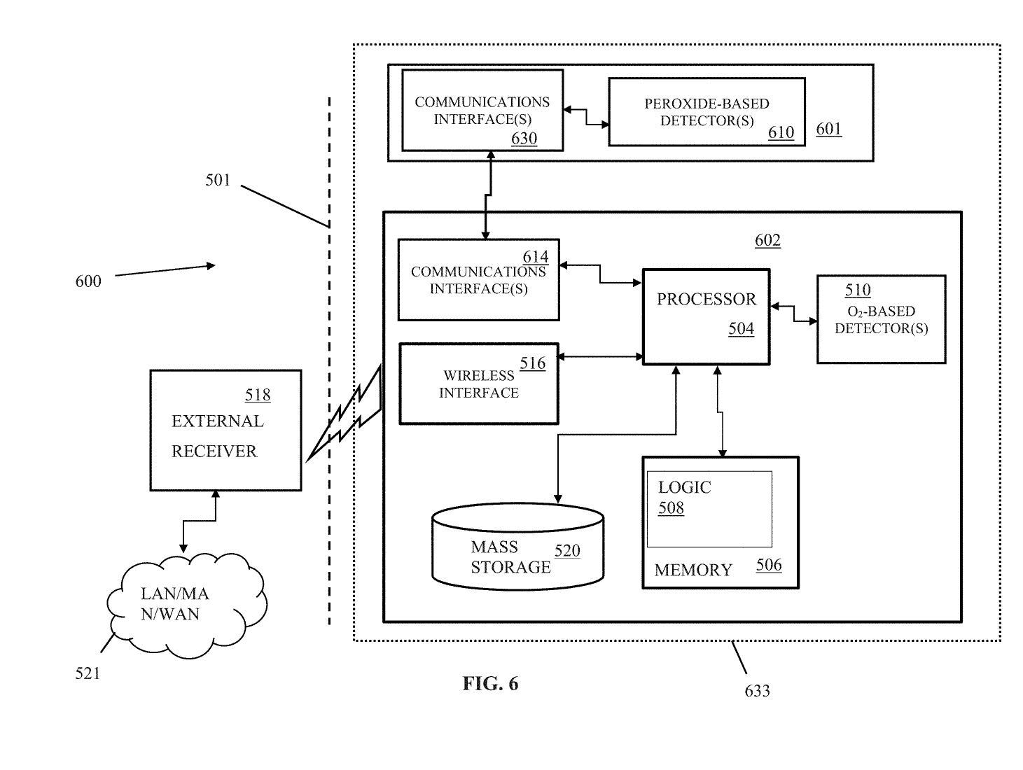

FIG. 6 is a functional block diagram of another embodiment of an implantable heterogeneous sensing device and associated receiver apparatus architecture 600 according to the disclosure, wherein the device comprises a first portion 601, and a second “add-on” module 602 that can be mechanically, logically, and/or electrically coupled to the first portion. For instance, the first portion may comprise a third-party implant device that includes a communications interface 630 (e.g., wireless interface, micro-USB port, multi-pin connector/cable interface, IrDA interface, or other) whereby data can be transferred between the first portion 601 and the module 602. In the illustrated embodiment, the module 602 acts in effect as an intermediary and proxy for the first portion 601 with respect to the external receiver 518, as well as a calibration/confirmation/correction entity as described elsewhere herein. Specifically, as shown, the exemplary embodiment of the module 602 obtains signals/data generated by the first detector(s) 610 of the first portion 601 via the respective communications interfaces 630, 614, and then operates in a similar fashion to the architecture of FIG. 5 thereafter; i.e., whether via on-device 602 processing, off-device (e.g., receiver 518 or other entity in communication therewith) processing, or combinations thereof, as described subsequently herein with respect to FIGS. 9-11.

One feature offered by the architecture 600 of FIG. 6 is that two heterogeneous and discrete devices (with respective different analyte detector types) can be mated (i.e., at least placed in signal communication via the interfaces 630, 614) so as to provide the enhanced functionality in terms of calibration, accuracy, and implant longevity described elsewhere herein.

In one variant, the devices 601, 602 can be physically mated and implanted as a unitary device 633, or implanted separately (e.g., in proximity to one another using same incision and pocket) and communicate wirelessly within the host's body, in addition to the wireless communication between the module interface 516 and external receiver 518.

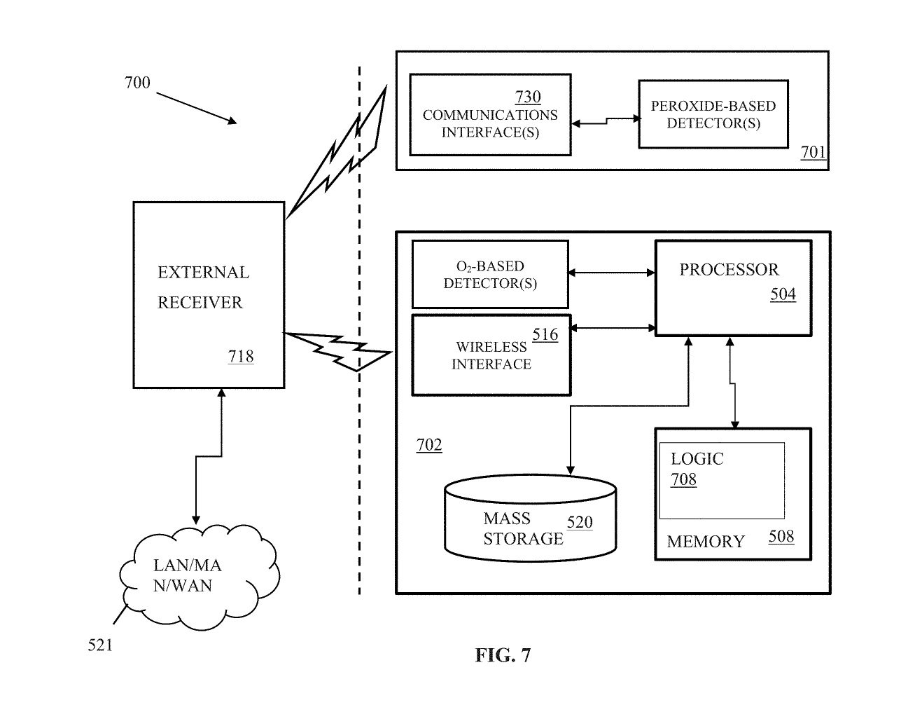

FIG. 7 is a functional block diagram of yet another embodiment of an implantable heterogeneous sensing device and associated receiver apparatus architecture 700 according to the disclosure, wherein the receiver 718 is configured to receive signals from each of the heterogeneous detector elements (and their host platforms 701, 702) directly. In contrast to the architecture 600 of FIG. 6, the architecture 700 of FIG. 7 obviates the need for a communications interface between the two implanted devices 701, 702, and instead uses the (indigenous) wireless interfaces 730, 516 of the respective devices 701, 702 to communicate signals/data obtained from their respective analyte detectors to the external receiver 718, the latter having the capability of wireless communication with each different device via its prevailing communications protocol and air interface type. For instance, one device may use a given RF frequency and modulation and coding scheme (MCS), while the other device may use a different frequency and MCS.

### Receiver Apparatus

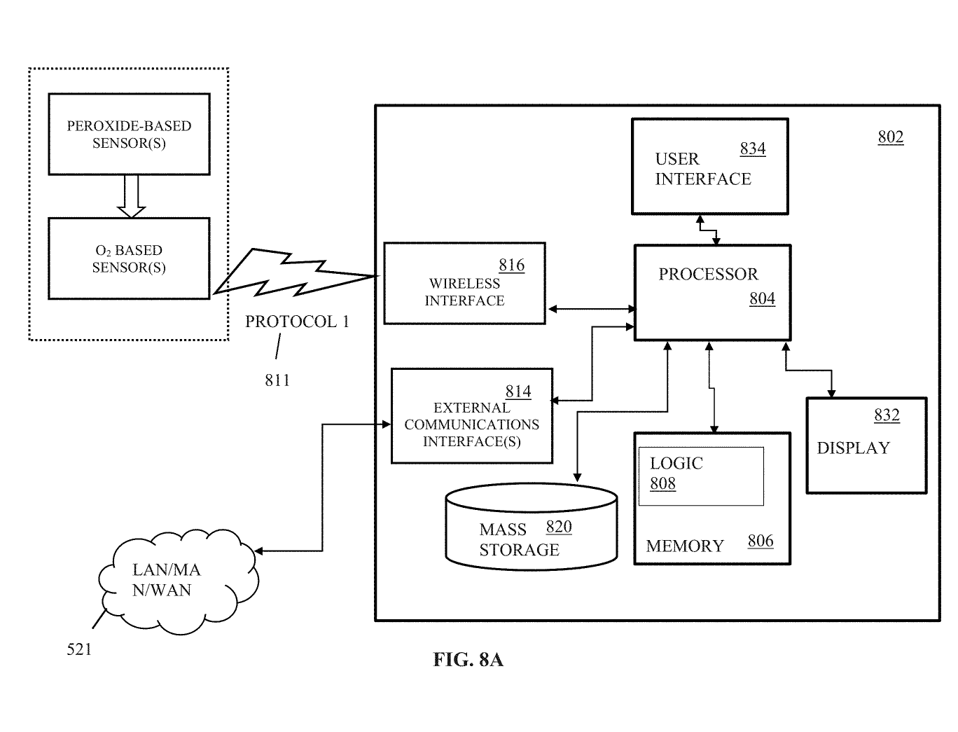

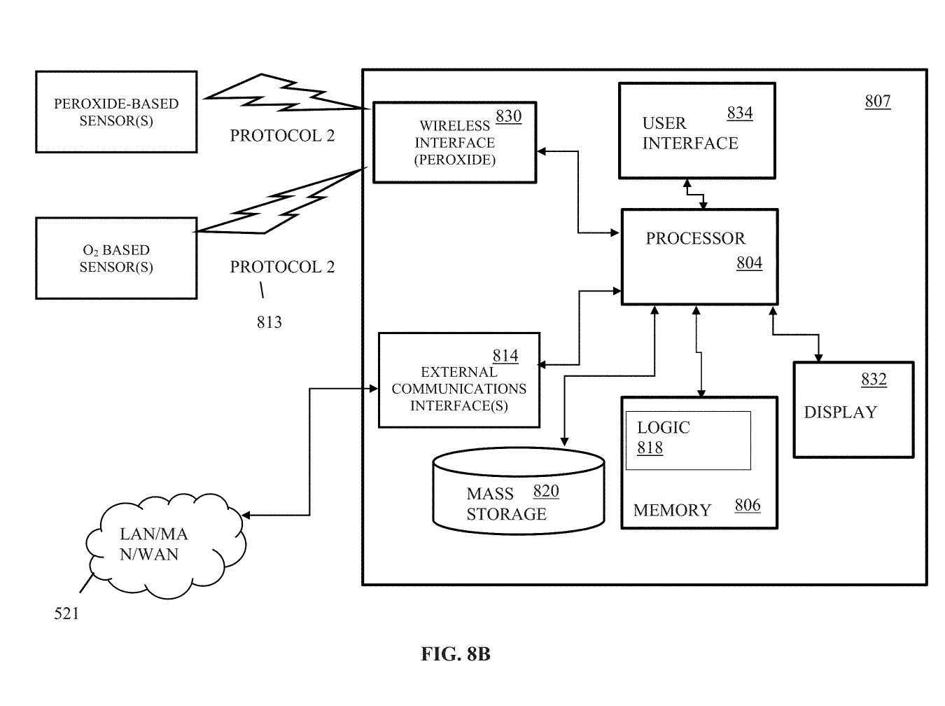

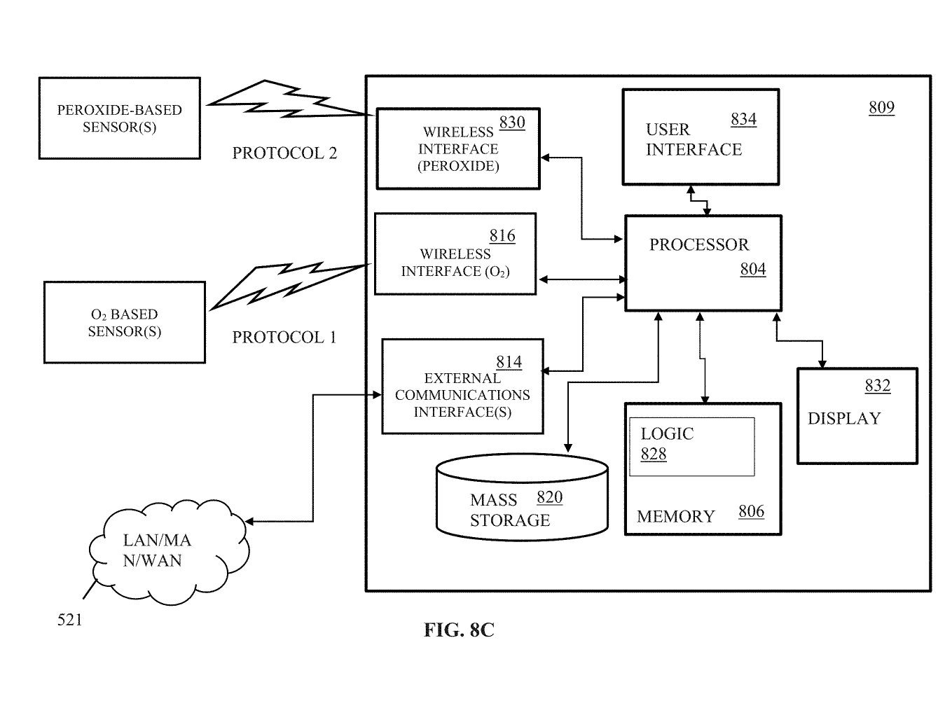

Referring now to FIGS. 8A-8C, various embodiments of the receiver apparatus 518, 718 shown in FIGS. 5-7 herein are described in detail.

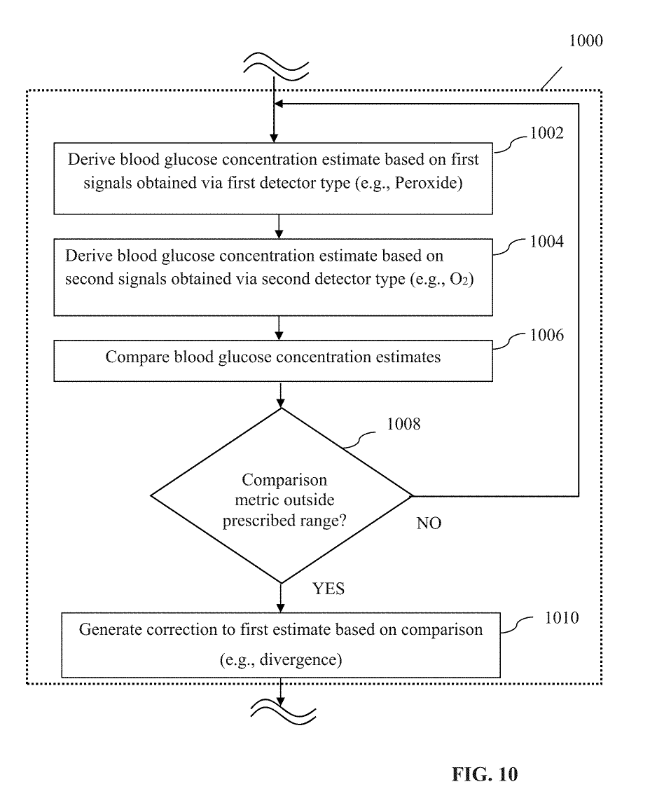

FIG. 8A is a functional block diagram showing one embodiment of the wireless receiver apparatus 802, wherein the heterogeneous detector types (e.g., peroxide-based and O2-based) utilize a common wireless interface 816 of the receiver 802 to obtain the peroxide-based detector signals and the O2-based detectors signals (whether aggregated or independent of one another). For example, in one exemplary implementation, the protocol 811 used to communicate between the in vivo device(s) (e.g., the sensor device 300 of FIG. 4) and the receiver 802 comprises an indigenous wireless protocol utilized by the O2-based sensor (e.g., a 433 MHz wireless signal that is modulated with data according to a prescribed modulation type and data encoding format). In such case, the source data generated by the two heterogeneous detector types can be segregated into packets or other data structures specific thereto, so that the individual source data can be preserved. Hence, in one variant, the logic 808 operative to run on the receiver (e.g., software “app”, firmware, etc.) which is normally configured to receive and process the O2-based detector data is enhanced so as to also receive and process the peroxide-based sensor data; e.g., for: (i) purposes of comparison; (ii) communication to a cloud-based entity (or another receiver) as a pass-through for the peroxide-based data; and/or (iii) use of the peroxide-based sensor as a primary source of data (with the oxygen-based sensor used in a confirmatory or calibration capacity, as described in greater detail below with respect to FIGS. 9-10).

As shown, the receiver apparatus 802 may also include other functionality, such as a communications/data interface 814 for communication with external devices and/or networks 521, a mass storage device 820 for storage of received data, a processor apparatus 804 for, inter alia, execution of the computerized logic 808, a memory device 806 (e.g., program and/or data memory), a display device 832, and a user interface 834.

Alternatively, the receiver 807 of FIG. 8B is configured to use the protocol 813 associated with the peroxide-based sensor and its associated interface 830, such as where the logic 818 operative to run on the receiver 807 is configured to ordinarily utilize the peroxide-based data for generation of e.g., blood glucose level(s), yet is also capable of receiving O2-based sensor data that are “piggybacked” onto the existing data transfer protocol (e.g., through inclusion in packets that are inserted into the transmitted data stream in gaps or otherwise unutilized resources of the protocol 813), and even utilize the received piggybacked data for e.g., confirmation, calibration, comparison, etc. As a simple example, the O2-based sensor data may be inserted into packets compatible with the protocol 813 (e.g., of a standardized type and format) by the O2-based sensor processing logic 708 (FIG. 7), and transmitted via the protocol 813 during idle periods on the air interface (e.g., when the peroxide-based sensor apparatus is not transmitting), so as to avoid mutual interference or collisions at the receiver 830, in effect forming a time-division multiplexing scheme. Other approaches may be used as well, such as (without limitation) spread-spectrum multiple access (e.g., FHS SS or DSSS), frequency-divided multiple access (FDMA), and orthogonal frequency division (OFDM). A carrier sense with collision detection scheme (e.g., CSMA/CD) may also be utilized, such that the O2-based device need not have any a priori knowledge regarding timing of the protocol 813 or scheme used by the (host) peroxide-based interface, but rather merely backs off when collisions are detected, and retries the transmissions until completed.

FIG. 8C is a functional block diagram of yet another embodiment of a wireless receiver apparatus 809, wherein two heterogeneous protocols 811, 813 are used for the respective sensor devices to communicate with different wireless receivers 816, 830 of the receiver apparatus 809. In this instance, the receiver acts in effect like a “dual mode” device, capable of simultaneous (or near simultaneous, depending on wireless frequencies and modulation and coding schemes (MCS) of the two interfaces 816, 830) reception of both the peroxide-based and O2-based detector data. The logic 828 running on the receiver 809 is configured to utilize both types of data for any number of functions, including for instance comparison, pass-through, confirmation/calibration as discussed supra, and without any direct communication between the two heterogeneous detectors themselves.

### Methods of Operation

Referring now to FIGS. 9-10, various methods of operating the heterogeneous sensor apparatus and receiver apparatus of the present disclosure are described.

FIG. 9 is a logical flow diagram illustrating one embodiment of a generalized method for use of heterogeneous detectors for measurement of an analyte (e.g., blood glucose) within a living being. As shown in FIG. 9, the method 900 generally includes first enabling the heterogeneous detector device(s) per step 902. In one case, the two devices are in fact part of a common device, such as the device 300 of FIG. 4, and the enabling comprises placing the device 300 in an operational state where it can obtain data (i.e., via “deep tissue” implantation as described in application Ser. No. 14/982,346 previously incorporated herein).

Next, per step 904, signals from the first detector type (e.g., peroxide-based) are obtained. As used in the present context, “signals” may include (without limitation) “raw” digital data, processed (e.g., filtered, normalized or compressed) digital data, raw or processed analog signals (e.g., variations in measured voltage), or yet other forms of information.

Per step 906, corresponding signals are obtained from the second detector type (e.g., O2-based). Notably, the obtained signals from each detector type of steps 904 and 906 may also be heterogeneous, and need not be in any way similar or commonly formatted, although it will be recognized that use of a common format may enhance subsequent processing.

Per step 908, the accuracy or metric of interest of the first signals is evaluated based at least on the second signals. In the exemplary implementation, the first detector type (peroxide) is known to be less accurate over time due to drift and other confounding phenomenon (resulting from, inter alia, accelerated FBR as described above), and hence the second detector-type signals are used as a confirmation or calibration of the first signals.

Per step 910, the need for correction of the first signals based on the evaluation of step 908 is determined (as described in greater detail below with respect to FIG. 10), and a correction that can be applied to the first signals generated (where needed) per step 912. The generated correction is then applied to the first signals per step 914 of the method.

It will be appreciated that the types and/or mechanism by which the correction is applied per FIG. 9 are in no way limited to any particular point within the derivation of the ultimate detector “output” (e.g., indicated numerical blood glucose level). Specifically, the present disclosure contemplates application of such correction of the data in more raw states (e.g., where a constituent voltage and/or current are used as a basis of deriving the ultimate blood glucose level output, the voltages/currents can themselves be corrected, thereby propagating the change down the estimate derivation processing chain). However, such approach generally requires a more intimate knowledge of how the output (estimate of blood glucose level) is actually determined within the first detector type, which may not be readily available. Hence, the present disclosure also contemplates other approaches which require no such intrinsic knowledge; rather, where the outputs are “apples to apples” (e.g., mmol/L or mg/dL), the correction can simply be added or subtracted (or multiplied) to the first detector type output. Intermediate points in the derivation processing chain can be used as well; e.g., where an intermediary/penultimate quantity or parameter is produced by the first detector type (before conversion to the ultimate output) for application of the correction. Various other schemes will be appreciated by those of ordinary skill in the signal processing arts when given the present disclosure.

FIG. 9A is a logical flow diagram illustrating one exemplary implementation of the generalized method of FIG. 9. In the method 920 of FIG. 9A, the device (such as the device 300 of FIG. 4) is implanted within the host per step 922.

Steps 924 and 926 obtain the first detector type signals and the second detector type signals, respectively.

Per step 928, the two obtained signals are aggregated (e.g., by the processor apparatus 504 and associated logic 508 shown in FIG. 5), and transmitted to the receiver apparatus (see e.g., the apparatus 802 of FIG. 8A).

The transmitted signals are received at the receiver per step 930, and the computerized logic (e.g., software/firmware) 808 of the receiver is utilized to evaluate the need for correction, confirmation, and/or calibration for the first detector type signals using at least the received second detector type signals contained within the aggregated data. In this implementation, the in vivo (implanted) sensor device does not have the on-board intelligence/processing capability to determine the correction, calibration, etc. in vivo, and hence the receiver apparatus and its logic 808 are used to provide this functionality.

FIG. 9B is a logical flow diagram illustrating another exemplary implementation of the generalized method of FIG. 9, wherein two separate interfaces (or one common yet virtually divided interface) are used, with respective communications protocols, to transmit the first detector type and second detector type data, respectively. Per step 947 of the method 945, the devices (which may be aggregated or combined as in the device of FIG. 4, or separate, including one implanted and one not), are enabled for operation.

Per steps 949 and 951, the respective signals are obtained, and then transmitted over their respective communications interfaces per step 953 to the receiver apparatus (see e.g., receiver 809 of FIG. 8C herein). Per step 955, the respective signals are each received and processed by the logic 828 of the receiver 809 at steps 957, 959, 961, and 963 in order to determine the need for correction or calibration, and to apply any such correction or calibration to the output.

Notably, in this embodiment, there is no communication between the first and second detector types, or “mixing” or common analysis of the signals until after receipt by the receiver 809. Accordingly, the two respective indigenous communication protocols of the devices can remain intact, thereby obviating any protocol or other modifications to the devices or their firmware/interfaces. Rather, it is only the logic 828 of the receiver which ultimately makes use of the two signal sets. Further, so as to obviate having to have a common receiver with the two heterogeneous interfaces/protocols, the present disclosure further contemplates provision of the received data/signals relating to each detector type (received via, e.g., proprietary devices of the respective manufacturers) to a common processing entity (e.g., smartphone, PC, tablet, etc.) via back-end communication interfaces of the receivers (such as Bluetooth or Wi-Fi or USB), the common processing entity containing the logic 828 to utilize both signals for calibration/correction/confirmation as described elsewhere herein, such as via a downloadable software app or the like, or connection with a cloud processing entity 521.

FIG. 9C is a logical flow diagram illustrating yet another exemplary implementation of the generalized method of FIG. 9, wherein a supplemental “module” such as that shown in FIG. 6 herein is utilized. Per step 972 of the method 970 in FIG. 9C, the “module” (e.g., one containing the second detector type) of FIG. 6 is mated to the host device; e.g., an extant peroxide based implantable device. As used herein the term “mated” refers to a logical or signal connection between the two devices, which may be wired or wireless in nature, although the two devices may also be physically and/or electrically mated (or even one enclosed within the other) if desired. Per step 974, the first detector type (e.g., peroxide-based) of the host device is used to obtain signals of the type previously described; these obtained signals are then transmitted via the aforementioned logical coupling to the module (with second detector type) per step 976. Complementary signals are obtained by the second detector type of the module per step 978, and the two signals are used for the evaluation of corrections or calibrations to be applied per steps 980, 982, 984, and 986 of the method 970.

Notably, at step 988, the “corrected” first signals are transmitted to a receiver associated with the host device using e.g., an extant wireless protocol of the host, yet using the wireless interface of the module. This approach obviates the need for any special software “app” or firmware in the receiver. In one implementation, the transmitter of the host is put to sleep (e.g., put into a dormant state through, e.g., assertion of a logic “high” on an appropriate control input on the transmitter) so as to preclude interference with the module transmitter. Any access or scrambling codes of the host transmitter may also be passed to the module transmitter (via the logical connection between host and module according to a wireless or data bus protocol) for use with the host-compatible receiver.

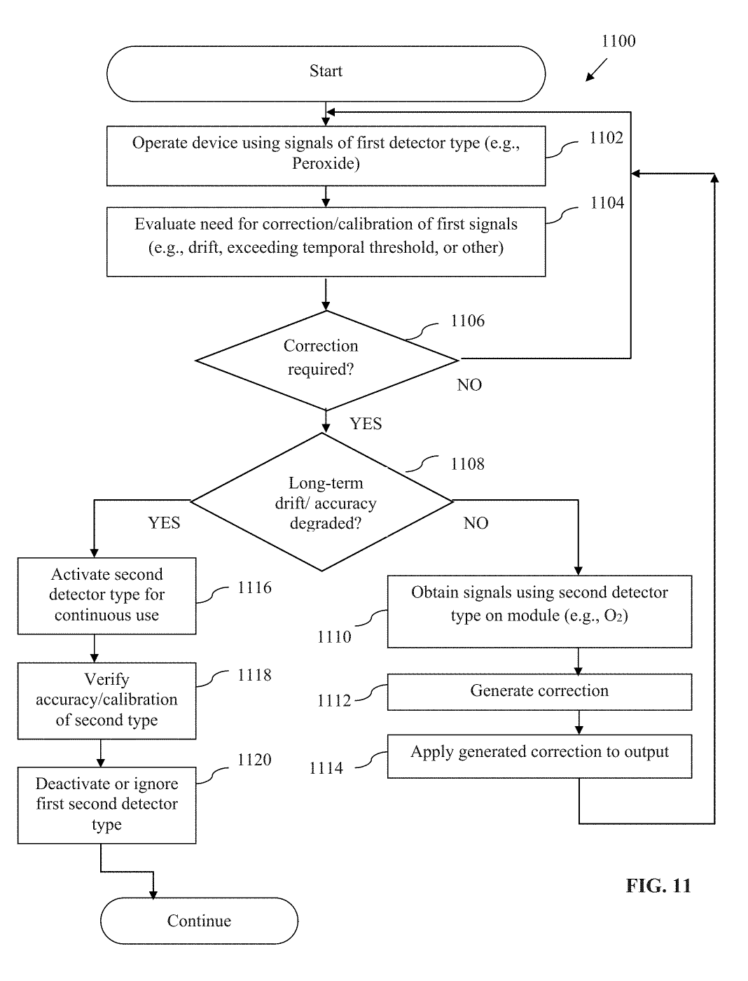

FIG. 10 is a logical flow diagram illustrating one exemplary implementation of the evaluation and correction processes (i.e., outlined method 1000) of the method of FIG. 9. As shown in FIG. 11, the method 1000 includes first deriving an estimate of the level of the analyte of interest (e.g., blood glucose concentration) based on the signals of the first (e.g., peroxide-based) detector(s) per step 1002.

Next, per step 1004, a comparable estimate is derived based on the second detector type (e.g., O2-based).

Per step 1006, a comparison of the two estimates is performed. In one variant, the comparison includes: (i) normalizing the estimates to a common measurement system (e.g, mg/dL) and reference level, and (ii) conducting a difference of the two normalized estimates). It will be appreciated that any number of different comparison techniques may be used consistent with the method 1100, including mathematical averaging of groups of estimates, statistical analysis (e.g., determination of mean, median, σ (standard deviation), σ² (variance), least-squares fitting, and others) may also be used to make a meaningful comparison of the data generated by the two differing detector types.

Per step 1008, the results of the comparison of step 1006 are then evaluated against one or more metrics or criteria, such as a prescribed allowable range or magnitude of difference between the estimates of the two detector types. Note that these metrics or criteria may also be: (i) dynamic (e.g., temporally variant, or variable based on the value of analyte concentration, such as where a “tighter” band is desired at one concentration as compared to another due to potentially more severe deleterious effects to the host being if the estimate is significantly in error); and (ii) asymmetric (e.g., a “higher” estimate error may be less sever/deleterious than a “lower” estimate error).

If the metric or criteria evaluation fails (e.g., is outside the prescribed range, then a correction to the first (e.g., peroxide-based) estimate is generated and applied thereto per step 1010.

In another variant (not shown), the process 1000 is configured to iterate; i.e., await the results of various passes through the logic loop of FIG. 11 before any correction is applied (or before a complete correction is applied), so as to obviate “hunting” or feedback-loop error propagation due to, e.g., the host being's actual blood glucose concentration being estimated while in a state of high variability (as opposed to a more stable state).

### Adaptation Circuitry and Methods

In some cases of implantation, the FBR and/or fibrosis phases of wound healing may block or cover one or more of the sensor elements 206,306. The sensor apparatus, however, includes multiple sets of sensing and reference sensing elements, which are in one implementation adapted to dynamically compensate for e.g., FBR, fibrosis, or other so-called “confounding factors” (described in U.S. Pat. No. 7,248,912, previously incorporated herein) occurring proximate the sensing elements, thereby maintaining the accuracy of the device as a whole. Specifically, the sensor apparatus 200,300 may have the advantage that the active sensor reading is compared to the reference sensor for glucose detection (i.e., “hydrogen peroxide-sensing differential measurement” and “oxygen-sensing differential measurement”, described supra). Thus, if the active sensor is blocked by foreign body giant cells, granulation tissues, and/or fibrous host tissue, it is likely that the adjacent reference sensor is also blocked. Readings from the sensing element pair will indicate that they are non-functional and should be excluded from determining the diabetic patient's glucose level.

The sensor apparatus 200,300 has the further advantage that if one or more pairs of sensors are non-functional, the glucose level may be determined from the remaining sensor pairs. Accordingly, as sensing elements or sets thereof become inoperative or unreliable, these elements/sets can be selectively removed from the signal processing logic and deactivated while other sensor pairs remain active. Alternatively, the weight of any signals generated by such compromised elements or pairs may be reduced over time so as to progressively reduce their contribution to the “composite” signal generated by the device. Furthermore, if one of the sensor element types (i.e., the hydrogen peroxide-based sensor element type or the oxygen-based sensor element type) becomes unreliable due to operability and/or physiological conditions these elements/sets of a specific type can be selectively removed from the signal processing logic and deactivated while other sensor pairs (of the other sensor element type) remain active such that the glucose level may be determined from the remaining sensor pairs.

Moreover, the aforementioned ability to remove or reduce the contribution of a given detector element or pair enables compensation for detector failure due to, e.g., leakage or other fault. As noted elsewhere herein, the exemplary sensor apparatus maintains the regions of each detector contacting the host's solid tissue enzyme-free (both through use of the non-enzymatic membrane 277 (see FIGS. 3, 3B) and manufacturing processes which avoid contamination of the spout region with the enzyme matrix of the cavity), and electrically insulated. However, in the case of a manufacturing defect, failure of a component (e.g., non-enzyme membrane 277 or outer membrane 230), or other such occurrence, the tissue response in a region localized to that (failed) detector element may increase due to the presence of the enzyme, electrical stimulation, etc., which can result in degradation of the performance of that particular detector element (if not already degraded due to component failure). By identifying such failures or tissue responses, the affected detector(s) can be electrically removed from further signal processing while the sensor 200, 300 is implanted.

Exemplary apparatus and methods for evaluating and adjusting operation of an implanted analyte (e.g., glucose) sensor which may be used consistent with the present disclosure are described in U.S. Pat. No. 7,248,912 to Gough et al. issued Jul. 24, 2007 and entitled “Tissue Implantable Sensors for Measurement of Blood Solutes”, previously incorporated herein, although it will be appreciated that other apparatus and methods may be used alternatively or in addition to those described in U.S. Pat. No. 7,248,912.

In one implementation of the sensor apparatus 200, 300, the adaptation circuitry (logic) of the apparatus is configured to (i) detect degradation of the first type of detector (e.g., peroxide based), such as with time; and (ii) adjust the operation of the sensor apparatus to account for the degradation, such as via more frequent calibrations/corrections, confirmation, and/or retirement of the first detector type in favor of the second detector type (e.g., oxygen-based) so as to, inter alia, extend the implanted longevity of the apparatus. FIG. 11 herein illustrates one embodiment of a method of operating the sensor apparatus (e.g., that FIG. 2 or 4) in such fashion.

As shown in FIG. 11, the method 1100 includes operation of the device using the first type of detector (e.g., after implantation) per step 1102. When one or more criteria for evaluation of the output of the first detector elements is met (e.g., expiration of a prescribed time period, assessed “drift” in the output, presence of other artifacts in the output signals, and/or other) per step 1104, the need for correction of the output is evaluated per step 1106. If no correction is required (e.g., the temporal period has expired, but the first detector elements are still operating within parameters), the method 1100 returns to operation (and subsequent evaluation per step 1104).

If, however, correction is required, the logic of the sensor apparatus (or external receiver device, or both in combination) is utilized in step 1108 to evaluate whether the triggering criterion/criteria of step 1104 is in fact related to or indicative of a long-term degradation process, or “catastrophic” event that degrades accuracy of the first detector elements. For instance, as described supra, FBR that has occurred over time (due to e.g., the presence of the peroxide-based enzyme in the exemplary first detector type) may have progressed to the point of significantly affecting the operation of the peroxide-based detectors. Advantageously, the exemplary oxygen-based detectors do not suffer from the same magnitude of FBR, and hence even on a common platform such as the apparatus 200, 300 of FIGS. 2-4, the secondary detectors can still function accurately well beyond the point that the primary (peroxide-based) detectors can.

Hence, if such degradation of the first or primary detectors is occurring per step 1108, the method 1100 proceeds to step 1116, wherein the secondary (e.g., O2-based) detectors are activated for full-time use as the primary detectors (versus just being used in a calibration or corrective capacity). Once activated, the second detectors are verified for calibration/accuracy (step 1118), and then used to logically replace the first detectors within the circuitry, and the first detectors electrically deactivated (although, as noted in FIG. 11, the first detectors may continue to operate and merely be ignored as a viable signal source).

Alternatively, if the assessment of step 1108 indicates that no long-term or catastrophic degradation of the first detectors is detected, the necessary correction to the first detector output signals is generated and applied per steps 1110, 1112, 1114, after which the method 1100 returns to continue operation with the first detectors used as the primary output signal source.

It is also appreciated that the exemplary oxygen-based detectors of the sensor apparatus 200,300 are also advantageously insensitive to interfering or confounding substances; e.g., low molecular weight species such as acetaminophen (e.g., Tylenol®-C₈H₉NO₂; molecular weight 151.16). As is known, while the glucose oxidase enzyme is highly specific to the glucose molecule, migration of acetaminophen through to the sensing electrodes can adversely impact operation of an implantable glucose sensor, such adverse impact being severe enough to warrant contraindication of acetaminophen for the host during monitoring. Contraindication of such a common pain reliever is highly undesirable from a practical standpoint; the host must strictly utilize an alternate over-the-counter pain reliever which is not contraindicated.

Moreover, one errant ingestion by the host during monitoring (e.g., mistakenly swallowing acetaminophen versus a non-contraindicated substance) can cause significant errors in the estimated blood glucose level, often in a non-conservative direction which can even be life-threatening to the host (i.e., erroneously indicating that the subject has a greater blood glucose level than they actually do, and either causing the host to treat the erroneously-elevated glucose level with glucose-lowering medication or avoid taking action which could otherwise mitigate an actual low blood glucose condition).

Hence, the exemplary apparatus 200, 300 provides yet another benefit from the standpoint that the logic or adaptation circuitry (when on-implant or external thereto) can be configured to selectively disable or ignore signals from the first (peroxide-based) detectors which are compromised due to confounding substances, at least temporarily.

In yet another embodiment, the implantable sensor apparatus described previously herein (e.g., one comprising one or more peroxide-based detectors and also one or more oxygen-based detectors) is operated such that the peroxide-based detectors are utilized as the primary signal source immediately after implantation of the sensor apparatus and for a period of time thereafter, so as to permit for stabilization and/or other operational “optimization” of the oxygen-based detectors. Specifically, such a period of time may be needed to, inter alia, ensure that healing has occurred to an extent sufficient for both the “main” or primary enzyme-containing detectors (of the oxygen-based detectors) and their associated oxygen reference detectors to achieve stable contact with the tissue, such that both are exposed to the same or similar “micro-environment.” Specifically, variations in the local spatial and electrochemical environment to which the individual detector elements are exposed may exist immediately after implantation, and for at least some time thereafter. Some of these variations may exist due to ongoing wound healing processes, FBR, or yet other factors; for instance, complete contact between the active portion of a given detector element and the surrounding tissue may not yet exist. Hence, the environment to which the detector elements of the implanted sensor apparatus are exposed during this initial period may not be identical with those present during the remainder (and great majority) of the total implant period of e.g., 12 or even 18 or 24 months.

During this initial “settling” period, other types of sensors such as the aforementioned peroxide-based sensor, might not require such stable contact (because they do not use a reference detector that must be kept in the same microenvironment as the main detector, or for yet other reasons), and hence can be used to replace, augment, or supplement the operation of one or more of the oxygen-based detector elements of the sensor apparatus.

A number of different variants are envisaged under the foregoing model. For example, in one such variant, the elements of the first type of detector (e.g., peroxide-based) are operated immediately after implantation, without operation of the counterpart elements of the second type (e.g., oxygen-based) of detector. For instance, it may be known that the second type of detector will not produce sufficiently stable and/or accurate results until after a prescribed healing or other period, and hence there may be little utility in turning on such second detector elements during such period (which may also consume at least some of the electrical energy stored in the sensor apparatus battery). Accordingly, blood analyte level measurements in such a variant are provided solely by the e.g., peroxide-based detector elements during the initial settling period.

Alternatively, the oxygen-based detector elements may be run in parallel with the peroxide-based elements during the settling period, with the operation of the oxygen-based elements monitored (e.g., by software or firmware algorithms or other such logic operative to run on the implanted sensor apparatus, and/or an external receiver and processing device) to, for example, observe expected convergence of the stability or other operational characteristic of the oxygen-based detectors during the period, and hence confirm their suitability to act as the primary signal source(s) for the blood analyte level measurement.

As yet another alternative, the oxygen-based detector elements may be used as the “primary” blood analyte level measurement source during the settling period, with the (ostensibly) stable peroxide-based sensor acting in tandem as a confirmatory or bounding data source. For instance, the variation or degree of stability experienced by the oxygen-based detector elements during the settling period may be deemed acceptable for purposes of measuring the host's blood analyte levels, subject to (i) confirmation by the data produced by the peroxide-based detector elements; and/or (ii) averaging or other mathematical inclusion of the peroxide-based detector element signals with those of the oxygen-based detectors to, in effect, dilute the latter's contribution to the overall blood analyte level measurement produced by the sensor apparatus or receiver device during the settling period in case the oxygen-based detector element performance exceeds prescribed stability or variation limits during that period.

Likewise, the present disclosure also contemplates use of algorithmic processing which progressively more heavily weights the output of one detector element type (e.g., oxygen-based) over another (e.g., peroxide-based) over time, such that at a point in time after implantation, the “primary” signal is completely or almost completely composed of the signal derived from the first detector type.

It will be recognized that while certain embodiments of the present disclosure are described in terms of a specific sequence of steps of a method, these descriptions are only illustrative of the broader methods described herein, and may be modified as required by the particular application. Certain steps may be rendered unnecessary or optional under certain circumstances. Additionally, certain steps or functionality may be added to the disclosed embodiments, or the order of performance of two or more steps permuted. All such variations are considered to be encompassed within the disclosure and claimed herein.

While the above detailed description has shown, described, and pointed out novel features as applied to various embodiments, it will be understood that various omissions, substitutions, and changes in the form and details of the device or process illustrated may be made by those skilled in the art without departing from principles described herein. The foregoing description is of the best mode presently contemplated. This description is in no way meant to be limiting, but rather should be taken as illustrative of the general principles described herein. The scope of the disclosure should be determined with reference to the claims.

What is claimed is:

1. An implantable blood analyte sensing device, comprising:
at least one first analyte detector of a first type;
at least one second analyte detector of a second type differing from the first type; and
logic in signal communication with the at least one first analyte detector and the at least one second analyte detector, the logic configured to utilize signals generated by the at least one second analyte detector to enable at least one of: (i) confirmation of a blood analyte level estimate obtained from signals generated by the at least one first analyte detector; and/or (ii) calibration of a blood analyte level estimate obtained from signals generated by the at least one first analyte detector.

2. The sensing device of claim 1, wherein the at least one first analyte detector of the first type comprises a peroxide-based blood glucose detector, and the at least one second analyte detector of the second type comprises a non-peroxide based blood glucose detector.

3. The sensing device of claim 2, wherein the at least one second analyte detector comprises an oxygen-based detector having a glucose oxidase (GOX) and catalase enzyme matrix.

4. The sensing device of claim 2, wherein the non-peroxide based blood glucose detector comprises:
a biocompatible housing having a size and shape suitable for implantation in a body;
a plurality of non-peroxide based analyte detector elements;
circuitry operatively connected to the plurality of detector elements and configured to process at least a portion of signals generated by one or more of the detector elements to produce processed signals;
and
an electrical power source operatively coupled to at least the circuitry and configured to provide electrical power thereto.

5. The sensing device of claim 4, wherein said non-peroxide based detector elements each further comprise a membrane configured for direct contact with a biological tissue of a host being after implantation of the sensor device, the membrane at least partly permeable to diffusion of the blood glucose therethrough, yet which is configured to frustrate blood vessel ingrowth.

6. The sensing device of claim 4, wherein each non-peroxide based detector element further comprises at least one membrane configured for direct contact with a biological tissue of a host being after implantation of the sensor device, the at least one membrane at least partly permeable to diffusion of the blood glucose therethrough, yet which is configured to frustrate blood vessel ingrowth.

7. A method of operating an implantable sensing device, comprising:
utilizing at least a first detector element of the sensing device to determine blood analyte level within a host being; and
utilizing at least a second detector element of the sensing device to confirm the determined blood analyte level.

8. The method of claim 7, wherein the utilizing at least a first detector element of the sensing device comprises using at least one peroxide-based detector element, and the utilizing at least a second detector element comprises using at least one oxygen-based detector element.

9. The method of claim 8, wherein the confirmation comprises determining that the determined blood analyte level is within a prescribed range of values, the prescribed range based at least in part on a blood analyte level determined by the at least second detector element.

10. The method of claim 9, wherein the at least second detector element comprises a plurality of individual detector elements, and the blood analyte level determined by the at least second detector element is determined using at least an algorithm operative to at least weight signals produced by certain of the plurality of individual detector elements over signals produced by others of the plurality.

11. The method of claim 9, wherein the weighting of the signals produced by certain of the plurality of individual detector elements over signals produced by others of the plurality comprises weighting based at least on a detected calibration drift of one or more of the plurality over time.

12. The method of claim 7, wherein the utilizing at least a first detector element of the sensing device comprises using at least one oxygen-based detector element, and the utilizing at least a second detector element comprises using at least one peroxide-based detector element on an intermittent or periodic basis only so as to mitigate formation of a foreign body response within the host being.

13. A method of operating an implantable sensing device, comprising:
utilizing at least a first detection enzyme apparatus of the sensing device to determine blood analyte level within a host being; and
at least periodically utilizing at least a second detection enzyme apparatus of the sensing device to calibrate the determined blood analyte level;
wherein the second detection enzyme apparatus comprises an enzyme matrix which is configured to mitigate foreign body response within the host being through elimination of a substance or compound formed within the second detector enzyme apparatus, which is also used within the first detector enzyme apparatus for said determination of blood analyte level.

14. A blood analyte sensing device configured for implantation in a living host being, comprising:
at least one first analyte detector of a first type;
at least one second analyte detector of a second type differing from the first type;
a communications interface; and
logic in signal communication with the at least one first analyte detector and the at least one second analyte detector and the communications interface, the logic configured to process signals generated by the at least one first analyte detector and the at least one second analyte detector so as to enable transmission via the communications interface to a receiving apparatus external to the host being.

15. The sensing device of claim 14, wherein:
the at least one first analyte detector of the first type comprises a peroxide-based blood glucose detector, and the at least one second analyte detector of the second type comprises a non-peroxide based blood glucose detector.

16. The sensing device of claim 15, wherein the processing of the signals generated by the at least one first analyte detector and the at least one second analyte detector enables transmission via an extant communications protocol associated with the at least one first analyte detector.

17. A blood analyte sensing module configured for implantation in a living host along with another sensing device, the module comprising:
at least one analyte detector utilizing an enzyme matrix for detection of blood analyte levels;
a wireless communications interface; and
logic in signal communication with the at least one analyte detector and the wireless communications interface, the logic configured to process signals generated by the at least one analyte detector for transmission via the wireless communications interface to a receiving apparatus external to the host, the receiving apparatus associated with the another sensing device.

18. The blood analyte sensing module of claim 17, wherein:
the another sensing device comprises a peroxide-based glucose sensing device; and
the at least one analyte detector comprises an oxygen-based glucose detector; and
wherein the transmission via the wireless communications interface to a receiving apparatus external to the host being comprises transmission according to a wireless transmission protocol utilized by the peroxide-based sensing device to transmit signals to the receiving apparatus.

19. The blood analyte sensing module of claim 17, wherein the module is configured to physically mate to or integrate with at least a portion of the another sensing device to permit implantation of the module and the another sensing device as a unitary structure.

20. A blood analyte sensing module configured for implantation in a living host being along with another sensing device, the module comprising:
at least one analyte detector utilizing an enzyme matrix for detection of blood analyte levels;
a first communications interface;
a wireless communications interface; and
logic in signal communication with the at least one analyte detector, the first communications interface, and the wireless communications interface, the logic configured to process at least signals generated by the at least one analyte detector for transmission via the wireless communications interface to a receiving apparatus external to the host being, the receiving apparatus associated with the sensing module.

21. The blood analyte sensing module of claim 20, wherein the processing at least signals generated by the at least one analyte detector for transmission via the wireless communications interface comprises processing at least both: (i) signals generated by the at least one analyte detector; and (ii) signals generated by the another sensing device and received by the module via the first communications interface.

22. Wireless receiver apparatus configured to receive data from each of: (i) an implanted blood analyte sensing device; and (ii) another blood analyte sensing device, the receiver apparatus comprising:
a wireless communications interface; and
computerized processing logic in signal communication with the wireless communications interface, the logic configured to process at least (i) data generated by the implanted blood analyte sensing device and received via the wireless communications interface; and (ii) data generated by the another blood analyte sensing device and received via the wireless communications interface.

23. The receiver apparatus of claim 22, wherein:
the data received via the wireless communications interface from the implanted blood analyte sensing device are formatted according to a first communications protocol;
and the data received via the wireless communications interface from the another
blood analyte sensing device are formatted according to a second communications protocol different than the first communications protocol.

24. The receiver apparatus of claim 22, wherein:
the data received via the wireless communications interface from the implanted blood analyte sensing device are used to perform at least one of: (i) a calibration of the data received via the wireless communications interface from the another blood analyte sensing device; and/or (ii) confirmation of an accuracy of the data received via the wireless communications interface from the another blood analyte sensing device.
